(19) United States
(12) Patent Application Publication
(10) Pub. No.: US 2025/0325985 A1
(43) Pub. Date: Oct. 23, 2025

(54) Improved Microfluidic Chip, System and Method for Protein Purification

(71) Applicants: VIB VZW, Gent (BE); Vrije Universiteit Brussel, Brussel (BE)

(72) Inventors: Rouslan Efremov, Gent (BE); Gangadhar Eluru, Brussel (BE)

(21) Appl. No.: 18/870,428

(22) PCT Filed: May 26, 2023
(86) PCT No.: PCT/EP2023/064155

§ 371 Date: Nov. 28, 2024

(30) Foreign Application Priority Data
Jun. 1, 2022 (EP) 22176647.0

(51) Int. Cl. B01L 3/00; C07K 1/14; G01N 21/64; G01N 33/68

(52) U.S. Cl. CPC B01L 3/502761; B01L 3/502715; C07K 1/14; G01N 21/6486; G01N 33/6803; B01L 2200/0631; B01L 2300/0663; B01L 2300/0681; B01L 2300/0816; B01L 2300/087; B01L 2400/0439; B01L 2400/0487; B01L 2400/0622

(57) ABSTRACT

The current invention relates to an improved microfluidic device for protein/protein complex purification, as well as an associated methodology and system.

In particular it relates to systems and methods for electron microscopy, preferably cryogenic electron microscopy (cryo-EM), on protein samples, comprising: a. at least one microfluidic chip with inlet and outlet and purification device; b. at least one illumination means and detection means for, preferably fluorescence, measurements on the microfluidic chip; c. a pumping system adapted for operable connection to the microfluidic chip, and configured for controlling flow in the operably connected microfluidic chip; d. a microscopy grid, preferably a cryogenic electron microscopy (cryo-EM) grid, for holding fluid samples; e. preferably a cryogenic container, for a cryogenic coolant; f. preferably a transport system for moving the cryo-EM grid between a position for receiving a fluid sample from the microfluidic chip and the cryogenic container; g. a control system, preferably a processor, which receives information on the measurements from the detection means, configured for controlling the pumping system at least based on said information, and further configured for controlling the illumination and detection means and preferably also for controlling the transport system.

### CROSS-REFERENCE TO RELATED APPLICATIONS

This application is a national phase entry under 35 U.S.C. § 371 of International Patent Application PCT/EP2023/064155, filed May 26, 2023, designating the United States of America and published in English as International Patent Publication WO 2023/232662 on Dec. 7, 2023, which claims the benefit under Article 8 of the Patent Cooperation Treaty to European Patent Application Serial No. 22176647.0, filed Jun. 1, 2022, the entireties of which are hereby incorporated by reference.

### FIELD OF THE INVENTION

The present invention relates to a microfluidic chip for purification of proteins and/or protein complexes of interest from a sample, as well as an associated methodology and system. In particular, the invention relates to modular systems and methods for sample preparation for electron microscopy, preferably cryogenic electron microscopy (cryo-EM) on protein samples. Said system and methods comprising at least one microfluidic chip with inlet and outlet and purification device, wherein said chip provides for a customized setup of different sampling and purification modules. Said system for (Cryo-EM) microscopy sampling further providing for at least one illumination means and detection means for, preferably fluorescence, measurements on the microfluidic chip; a pumping system adapted for operable connection to the microfluidic chip, and configured for controlling flow in the operably connected microfluidic chip; preferably, a microscopy grid, preferably a cryo-EM grid, for holding fluid samples; preferably a cryogenic container, for a cryogenic coolant; preferably a transport system for moving the cryo-EM grid between a position for receiving a fluid sample from the microfluidic chip and the cryogenic container; a control system, preferably a processor, which receives information on the measurements from the detection means, configured for controlling the pumping system at least based on said information, and further configured for controlling the illumination and detection means and preferably also for controlling the transport system.

### BACKGROUND

Standard laboratory techniques of purification and (cryo) EM-grid preparation, that are employed for the structure determination of proteins, involve starting volumes in the order of few milliliters, due to the inherent limitation of the bulk purification columns and plunging devices used. Eukaryotic low copy proteins and complexes cannot be used in such large quantities due to their scarce availability, and this is one of the driving forces behind the strong increase in focus on microfluidics. As such, miniaturization is crucial in this field, but with currently available devices and under the procedures used currently, this remains highly time- and labor-intensive. It typically requires the operator to intervene multiple times throughout the purification, with these interventions being very meticulous actions, making the entire procedure cumbersome, notwithstanding preparatory work and actions after purification (for instance, deposition on a microscopy grid).

Especially in the field of cryogenic electron microscopy (cryo-EM), simplifying the procedural steps, from the original, unpurified sample, to the purified extract that is deposited correctly onto a cryo-EM grid, can potentially reduce the time necessary by a large extent, given the complexity of many of the substeps, and the desired throughput of sampling, each time with very low amounts of actual volumes in the samples. In the field of cryo-EM, sample preparation is a bottleneck in the workflow, being very time- and labor-intensive. Many known procedures used in the field require high amounts of supervision and very meticulous further activity between substeps. The current invention aims towards solidifying (the greater part of the) the entire procedure into a single action for the researcher, wherein the microfluidic chip then takes over the separate substeps, with minimal further requirements and activities for the researcher. Furthermore, given the miniaturization applied to the present invention, it allows the procedure to be scaled up and/or automized, making the process much more efficient, as well as requiring minimal volumes of the original sample to distill into a useful purified protein/protein complex.

There is a need for an automated microfluidic-based system that enables purification of the protein samples, using the underlying principles of standard purification techniques but integrated within a single microfluidic chip, preferably with means for subsequent deposition and plunging of EM grids.

Some devices have been developed in the field of microfluidics for purification of samples, but this is generally not specifically for protein/protein complex purification, and instead focus on nucleic acid purification. Furthermore, many of these devices still provide modular solutions, wherein the entire procedure, from an available minimal amount of a complex sample to providing purified native protein on a grid, cannot be performed on a single microfluidic chip. This means it still requires multiple interventions of an operator, and specifically time-intensive handling of ‘forwarding’ the resulting product from a first microfluidic chip to a second (and even a third, etc.) in order to undergo a further substep in the purification process.

### SUMMARY OF THE INVENTION

The present invention and embodiments thereof serve to provide a solution to one or more of above-mentioned disadvantages.

The present invention aims to provide for a stand-alone, microfluidic lab-on-a-chip product, with an associated methodology and system, where a low volume sample (in the order of microliters, preferably even lower) is provided to the chip, wherein it is purified aimed at a target protein and/or protein complex, even up to microgram amounts of purified protein, requiring minimal activities from an operator during the purification procedure, while also greatly reducing preparatory activities by the operator, and preferably also simplifying the procedure after purification (deposition and/or other steps). Moreover, the combination of providing an on-chip protein purification followed by a controlled application on a grid has significantly reduced the time required for sample preparation, resulting in an increased speed of the process time for cryo-EM applications allowing more sophisticated analyses with fewer starting material.

In a first aspect, the invention relates to an improved microfluidic chip for purification of one or more proteins and/or protein complexes of interest from a sample, specifically for use prior to cryogenic electron microscopy (cryo-EM) performed on purified proteins/protein complexes generated from the microfluidic chip. The chip herein comprises:
at least one chip inlet for receiving a fluid medium;
at least one chip outlet for dispensing fluid purified protein sample onto a microscopy grid, and preferably onto a cryo-EM grid;
a channel system or tubing-based structure extending between the outer parts of the chip application volume, specifically between the at least one chip inlet and the at least one chip outlet, said channel system internally comprising
a purification device or module for purifying one or more proteins or protein complexes of interest from a sample, preferably wherein the purification device comprises at least one inlet for introducing and/or for removing purification agents.

Said microchip may further integrate additional features providing solutions for current preparation of proteins for use in microscopy, preferably Cryo-EM microscopy. Said features include further purification devices for more profound purification strategies, one or more integrated detection systems or zones, one or more concentration modules for increasing the amount of protein per volume unit, and one or more on-chip valve systems, more specifically on-chip valve systems downstream of a purification device for controlling the fluid flow in the chip, and/or for allowing control of sample deposition from the chip outlet. In addition, a particular embodiments relates to a novel design of the integrated purification device, as to improve flexibility of on-chip purification. In what follows, more specific and preferred embodiments of the chip will be discussed.

In a second aspect, the invention relates to a system for electron microscopy, preferably cryo-EM, on protein samples, said system comprising:
at least one microfluid chip, preferably according to the first aspect of the invention;
at least one illumination means and detection means for, preferably fluorescence, measurements on the microfluidic chip;
a pumping system adapted for operable connection to the microfluidic chip, and configured for controlling flow in the operably connected microfluidic chip;
microscopy, preferably cryo-EM, grid for holding fluid samples;
preferably a cryogenic container, for a cryogenic coolant;
preferably a transport system for moving the cryo-EM grid between a position for receiving a fluid sample from the microfluidic chip and the cryogenic container;
a control system, preferably a processor, which receives information on the measurements from the detection means, configured for controlling the pumping system at least based on said information, and preferably further configured for controlling the transport system, illumination and detection means.

In what follows, more specific and preferred embodiments of the system will be discussed.

In a third aspect, the invention relates to a method for on-chip purification and/or enrichment of one or more proteins and/or protein complexes of interest from a sample, prior to cryo-EM, said method comprising the following step:
providing a protein sample at a chip inlet of a microfluidic chip, preferably a chip according to the first aspect of the invention, into a channel system of the microfluidic chip;
performing at least one purification step by means of a first purification device downstream of said chip inlet;
preferably dispensing the purified fluid protein sample onto a cryo-EM grid from a chip outlet via a deposition means downstream from the first purification device.

In what follows, more specific and preferred embodiments of the methodology will be discussed.

As can be seen from the above, the methodology and system allow for a broader application of the developed microfluidic chip beyond purely cryo-EM purposes, as will be discussed in the present document.

### DESCRIPTION OF FIGURES

FIG. 1 shows the minimum concentration of pure BSA that can be detected using our experimental set-up, with a plot of fluorescently tagged BSA at concentrations of 1 μg/μl, 0.1 μg/μl, and 0.01 μg/μl pumped alternately with buffer.

FIG. 2 shows a schematic overview of a system according to the invention.

FIG. 3 shows a schematic of a microfluidic chip according to the invention (dotted lines—1), attached to a pumping system configured for controlling flow in the chip, and the EM-grid and plunging arm of the system.

FIG. 4 shows the purification chromatogram observed during the GFP purification using the microfluidic chip.

FIG. 5A shows the atlas of the cryo-EM grid, FIG. 5B shows one of outermost squares from where data collection was done and FIG. 5C shows a typical image of the particles from one of the holes, for example 2.

FIG. 6A-D show the representative images and the reconstructed apoferritin for example 2.

FIG. 7 shows the β-galactosidase purification chromatogram for example 2.

FIG. 8A-D show representative images of grid, squares, holes, and the final reconstruction respectively for example 2.

FIG. 9A-9C shows a microfluidic chip represented in multiple layers (Layer 1-3).

FIG. 9A) Schematic of the designs involved in the fabrication of the chip; FIG. 9B) Zoom out image of top view of the layer-1 in a) near the entrance of the purification device, showing the pillar-based structure; FIG. 9A) Zoom out image of top view of the layer-2 in a) near the entrance of the purification device without pillar-based structure. The chip is produced by assembling layer 1 and 2 to produce a channel and pillar configuration of different heights near the entrance and exit of the purification device or column.

FIG. 10 shows the resulting signal strength (measured in Volts) with respect to total volume introduced into the microfluidic chip for 4 μl of cytoplasmic extract loaded onto the chip, in example 3.

FIG. 11 shows the fluorescent signal strength (measured in Volts) with respect to the introduced fluid in the microfluidic chip using the cytoplasmic extract from Escherichia coli expressing β-galactosidase under an extract-dye 5:1 (dotted line) and 40:1 (full line).

FIG. 12 shows the effect on the fluorescent signal strength (measured in Volts) under influence of detergent (0.2% dodecyl-maltoside (DDM), full line), or without detergent (dotted line).

FIG. 13 shows the fluorescent signal strength (measured in Volts) with respect to the introduced fluid into the chip, for the purification of β-galactosidase from a single colony of cells. 40 μl of cytoplasmic extract was obtained from a single colony of cells. 35 μl of this cytoplasmic extract was mixed with 1 μl of fluorescent dye and 25 μl of this was loaded onto the chip. Again, 1× and 1× amplifications were used during affinity and SEC respectively and 0.5 μl of affinity-purified protein was diverted into SEC column. The amount of β-galactosidase protein that is likely present in this amount of cytoplasmic extract that was pumped onto the chip, is about 0.5 μg (estimated based on reaction kinetics from spectrophotometer).

FIG. 14 shows a schematic representation of a cross-section of a channel at the specific site of entrance or exit of the purification device, where pillars are present (as in FIG. 9b). Hₚ, height of the pillar(s); H_{c}, height of the column (or purification device).

FIG. 15 shows a microfluidic chip (dotted lines—1), including protein concentration chambers and a delay line, said chip is attached to a pumping system configured for controlling flow in the chip, and the EM-grid and plunging arm of the system.

FIG. 16 shows an image of the SDS-PAGE analysis obtained from the on-chip purification of β-galactosidase, where the cytoplasmic extract was taken from a single colony of E. coli expressing β-galactosidase for Example 5.

FIG. 17A-17C shows the chromatogram (FIG. 17A) obtained for the complete purification of E. coli Complex-I from solubilized membranes using the microfluidic chip of the present invention; an image of the SDS-PAGE analysis (FIG. 17B) obtained from the collected SEC fractions collected after the microfluidic purification, and a micrograph (FIG. 17C) showing the Complex-I particles after the microfluidic purification and blotless EM-grid preparation.

FIG. 18A-18C shows different protein concentration experimental approaches. FIG. 18A) Schematic of a concentration methodology based on electrophoresis under anionic layer coating of the channel; FIG. 18B) Schematic of a concentration methodology that employs smaller affinity column; FIG. 18C) Schematic of a concentration methodology that uses a protein separation membrane.

FIG. 19 show the results of the protein concentration method for on-chip use by applying an electric field on an anionic bilayer. GFP protein sample (upper panels) or GroEL protein sample (lower panels) is introduced into the channel via the inlet and an electric field is applied. Images of the valve region at various time points during the experiment are shown: i) Valve region with buffer, ii) Valve region with protein prior to the application of electric field, iii) valve region with 14 times concentrated GFP in 4 nl after 80 sec of application of electric field, or 25 times concentrated GroEL in 4 nl after 160 sec of application of electric field, resp.

FIG. 20A-20B shows the setup and result of concentrating protein using small ion-exchange (IE) column. FIG. 20A) PDMS chip used for IE-based concentration illustrating all the key elements. The 2nd column was used for IE experiments; FIG. 20B) Chromatogram showing the signal during the concentration process, resulting in a sharp elution peak indicative of successful protein concentration.

### DETAILED DESCRIPTION OF THE INVENTION

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far as such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specify the presence of what follows e.g., component and do not exclude or preclude the presence of additional, non-recited components, features, element, members, steps, known in the art or disclosed therein. The terms may also encompass “consisting of” and “consisting essentially of”, which enjoy well-established meanings in patent terminology.

The terms “protein”, “polypeptide”, and “peptide” are interchangeably used further herein to refer to a polymer of amino acid residues and to variants and synthetic analogues of the same. A monomeric or protomer is defined as a single polypeptide chain from amino-terminal to carboxy-terminal ends. A “protein subunit” as used herein refers to a monomer or protomer, which may form part of a multimeric protein complex or assembly. The term “molecular complex” or “complex” refers to a molecule associated with at least one other molecule, which may be a protein (“protein complex”) or a chemical entity. The term “associating with” refers to a condition of proximity between a chemical entity or compound, or portions thereof, and a binding pocket or binding site on a protein. As used herein, the term “protein complex” or “protein assembly” or “multimer” refers to a group of two or more associated macromolecules, whereby at least one of the macromolecules is a protein. A protein complex or assembly, as used herein, typically refers to binding or associations of macromolecules that can be formed under physiological conditions. Individual members of a protein complex, such as protein subunits or protomers, are linked by non-covalent or covalent interactions. When a protein of interest is purified from a sample, the purified product is mainly composed of the monomeric protein, though a small fraction may still be present in a protein complex with another molecule. The “protein of interest” as used herein may however also refer to a ‘protein complex of interest’ if this is the desired outcome of the purification. The term “multimer(s)”, “multimeric complex”, or “multimeric protein(s) or assemblies” comprises a plurality of identical or heterologous polypeptide monomers. Polypeptides can be capable of self-assembling into multimeric assemblies (i.e.: dimers, trimers, pentamers, hexamers, heptamers, octamers, etc.) formed from self-assembly of a plurality of a single polypeptide monomers (i.e., “homo-multimeric assemblies”) or from self-assembly of a plurality of different polypeptide monomers (i.e., “hetero-multimeric assemblies”).

The term ‘complex sample’ as used herein refers to the complexity of a (biological) sample, such as a sample composed of living cell(s), single colony, mixtures of proteins, extracts, or biological samples obtained from an organism such as a blood sample.

A “microfluidic chip” is a set of micro-channels etched or molded into a material (e.g. glass, silicone or polymer). The micro-channels forming the microfluidic chip are connected together in order to achieve the desired features (mix, pump, sort, or control the biochemical environment). This network of microchannels trapped into the microfluidic chip is connected to the outside by inputs and outputs pierced through the chip, as an interface between the macro- and micro-world. The simplest current microfluidic device consists in micro-channels molded in a polymer that is bonded to a flat surface (such as a glass slide). The polymer most commonly used for molding microfluidic chips is PolyDimethylSiloxane (PDMS). PDMS is a transparent, biocompatible, deformable and inexpensive elastomer. It is easy to mold and bond on glass.

The term ‘cryogenic storage Dewar’, ‘cryogenic container’ or ‘Dewar’ as used herein, refers to a type of storage container suitable for storing cryogens (such as liquid nitrogen or liquid helium), whose boiling points are much lower than room temperature. Cryogenic storage Dewars may be a specialized type of vacuum flask, or may take several different forms including open buckets, flasks with loose-fitting

stoppers and self-pressurizing tanks. Dewars typically have walls constructed from two or more layers, with a high vacuum maintained between the layers. This provides very good thermal insulation between the interior and exterior of the Dewar, which reduces the rate at which the contents boil away. Precautions are taken in the design of Dewars to safely manage the gas which is released as the liquid slowly boils. The simplest cryogenic containers allow the gas to escape either through an open top or past a loose-fitting stopper to prevent the risk of explosion. More sophisticated cryogenic containers trap the gas above the liquid, and hold it at high pressure. This increases the boiling point of the liquid, allowing it to be stored for extended periods.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In what follows, the dispensing of the purified protein(s)/protein complex(es) of interest, which will also be referred to as the purified product, is discussed in relation to a microscopy grid, and often specifically a cryo-EM grid. It is of course also envisioned within the scope of the invention to provide deposits of the purified product onto or into other recipients, or even into or onto further processing devices. While the focus of the invention rests in its applicability in microscopy applications, it is not limited thereto.

Most microfluidic chips are substantially flat or planar, with the channel system meandering, as a serpentine channel, across the plane of the chip (length and width), but without changing in the height in the chip at which the channels extend, usually due to the production process, which is often by adding separate layers to each other, in which one layer comprises the channels. In what follows, the term “height” refers to the position perpendicular to the ‘plane’ of the microfluidic chip, in relation to the bottom surface of it. The ‘plane’ of the microfluidic chip is defined as the plane in which the channel system extends, which typically is parallel with a top and bottom surface of the chip. The terms length and width of the chip can be used interchangeably, as the dimensions in the plane of the chip.

In a first aspect, the invention provides to an improved microfluidic chip for purification of a protein of interest from a sample, said purification process preferably prior to cryogenic electron microscopy (cryo-EM) of the purified sample. The chip comprises at least the following:
at least one chip inlet for receiving a fluid medium;
at least one chip outlet for dispensing fluid purified protein sample onto a microscopy grid, preferably onto a cryo-EM grid;
a channel system extending between the at least one chip inlet and the at least one chip outlet, said channel system internally comprising a first purification device for purifying the protein of interest from a sample, preferably wherein the first purification device comprises at least one inlet for introducing and at least one outlet for removing purification agents;
wherein the chip comprises a deposition means for ejecting the resulting purified protein sample from the chip onto a material for further analysis, preferably onto a cryo-EM grid.

So in one embodiment, the microfluidic chip further serves for subsequent deposition of the purified protein/protein complex onto electron-microscopy grids, prior to cryogenic electron microscopy.

So a further preferred embodiment relates to a microfluidic chip for purification of one or more proteins and/or protein complexes of interest from a sample and subsequent deposition of the purified protein and/or protein complex onto electron-microscopy grid prior to cryogenic electron microscopy (cryo-EM), the chip comprising:
at least one chip inlet for receiving a fluid medium;
at least one chip outlet for dispensing fluid purified protein sample onto a cryogenic electron microscopy (cryo-EM) grid;
a channel system extending between the at least one chip inlet and the at least one chip outlet, said channel system internally comprising:
a first purification device for purifying one or more proteins or protein complexes of interest from a sample, preferably wherein the first purification device comprises at least one inlet for introducing or removing purification agents; and a deposition means controlled for ejecting the sample by a valving system for diverting the purified protein onto the electron microscopy grid to facilitate deposition.

In the field, very few microfluidic chips have been developed that can provide for a full purification and deposition process on a single chip, especially when desiring very low input volumes and high throughput speeds. Some chips that are commercially available, can perform certain substeps (for instance, purification chips), but do not have an outlet for dispensing the purified sample onto a microscopy grid, and are often missing other features. Furthermore, the majority of chips that have been developed, were developed for nucleic acid purification processes, but not customized for protein/protein complex purification.

Considering the specific application of the invention to cryo-EM purposes, it brings forth additional advantages. Cryogenic-EM grid deposition and processing is a delicate and time-consuming step, requiring the operator to withdraw or gather purified protein samples resulting from a preceding purification step, and to deposit this very accurately on the grid, which is then submerged into a cryogenic coolant. In the microfluidic chip of the present invention, the deposition means comprises a specific outlet present in the chip for dispensing the purified protein sample onto a microscopy grid. Said outlet can be modified and controlled to dispense sample volumes according to very accurate instructions, regarding volume, speed, etc., which frees up time for the operator.

The internal channel system comprises a purification device, which enforces the purification or enrichment of the protein or protein complex of interest from the sample. The type of the purification device can vary strongly, depending on the specific protein/protein complex of interest, a required minimal purification level, the desired processing speed, whether or not certain eluents may be used, or even available budget. Typical purification devices are discussed further on in the description, however, there is no limitation to which types are possible and which are not in the present invention, but known to the skilled person other than the potential for use in a microfluidic chip.

According to various embodiments, the purification device can be a column, a chamber, a channel, a well, a test tube, a capillary, or any other structure suitable for containing, retaining, or encapsulating a purification material or purification agent, diluent, and a fluid sample, preferably as present in the chip of the present invention. The purification device can contain a purification material or agent. The purification material can be any material that is capable of retaining or capturing a compound of interest from a sample on the purification device. Alternatively, the purification material can also be specifically chosen to retain or capture undesired compounds in the sample, thus allowing passage for the compounds, in particular the protein(s) or protein complex(es) of interest (i.e. acting in a flow-through mode rather than a capturing mode). The latter however requires a much higher level of knowledge on the undesired compounds in the sample, in order to eliminate all of these, and can require multiple purification devices to handle each of these undesired compounds. For example, the purification material can be a size-exclusion chromatography matrix, an affinity matrix, a gel-exclusion matrix, an ion-exchange resin matrix, size-exclusion, ion-exchange particles, hydrophobic interaction chromatography, a mixed-mode chromatography, or other materials capable of separation and purification of a fluid sample, or combination thereof. According to various embodiments, the purification material can be a powder, a particulate material, beads, a frit, a gel, a slurry, or a combination thereof, preferably compatible with the integration of the purification device in or on the chip. The purification material can be disposed in or loaded into the purification device in a dried form, sprayed into the purification device to adhere to the structure of the purification device, added to the purification device with a diluent, or loaded in any combination thereof.

According to various embodiments, the purification device can be a chamber that has a rectangular cross-section along the flow axis for the medium. An exemplary purification device can be about 0.50 mm deep, about 0.50 mm wide, and about 20 mm long, providing a total volume of about 5 microliters. Alternatively, the device may have a circular or a trapezoidal cross-section (along the flow axis). The purification device can accommodate volumes from about 1 nanoliter to about 75 microliters, but preferably has an internal volume ranging between 10 nanoliters and 10 microliters, and more preferably between 25 nanoliters and 5 microliters, or even between 50 nanoliters and 2.5 microliters. It should be noted that the volume typically varies depending on which type of purification device is used, as SEC (size exclusion columns, or gel filtration columns, as used interchangeably herein) for instance require a larger volume than affinity columns. According to various embodiments, the purification device can have the same height as the thickness of the substrate in which the purification device is formed, allowing the chip to produced very easily.

In a further improvement, the invention aims to introduce purification devices, and specifically affinity and size-exclusion columns or chambers with a strongly reduced volume (below 0.5 μl and below 5 μl respectively). The smaller volume is expected to provide smaller width to the detected elution peak (hence enhancing the concentration of the protein/protein complex). This also facilitates purifying smaller amounts of starting protein.

According to various embodiments, a purification material can be added to a purification device at manufacture, or before use of the purification device. The purification material can be saturated with a diluent. The purification material can be over-saturated with diluent so as to provide an excess diluent in the purification device. According to various embodiments, the purification material can be introduced into the purification device through an entrance opening, which is separate from the openings through which the purification device interfaces with the channel system.

The purification device comprises at least one inlet, but can optionally also comprise an outlet, for flushing out agents (washing buffers for instance). While it is also possible to remove such agents at further outlets downstream, it can be advantageous to perform this locally.

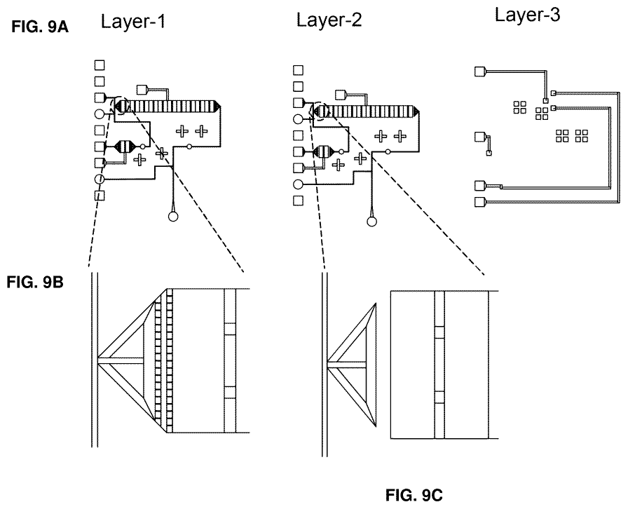

In a specific embodiment, the purification device is configured to withhold the purification materials, such as for instance beads, slurry, resin or other materials known to the skilled person. The design of said purification device is configured as for instance illustrated in FIG. 9, by assembling the chip from different layers of material, wherein the one layer has a different entrance and exit (or inlet and outlet) at the transition point with the channel.

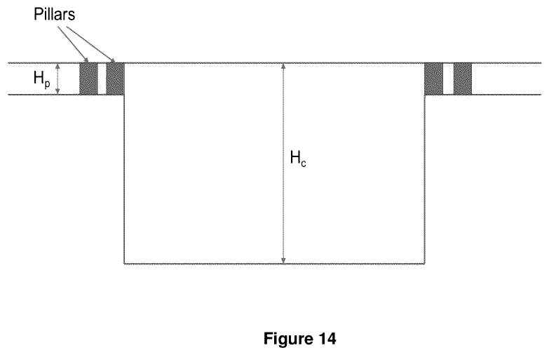

Specifically, one embodiment relates to the use of a pillar-based structure, as shown in FIG. 9 b and FIG. 14, in at least one layer of the chip, so as to allow entrance and flow out of the fluidic sample from the channels to pass through the purification device via the openings between the pillars. The presence of said pillar-based structures allows to retain the purification material present within the purification device. Generally, such pillar-based structures are of the same height as the purification device or column that is containing the purification material, though by introducing here a pillar-based structure in one or more layers, with a height of the pillars that is relatively lower to the total height of the column or purification device (see FIG. 14), this results in an additional benefit for the use of purification material with particle sizes that are smaller than the height of the purification device, but larger than the openings between the pillars.

In a preferred embodiment, the channel system comprises one or more channels, preferably with constant cross-section over its course. The channels transition into purification devices, detection zones and/or valve sections, and at said points typically widen or narrow in their cross-section.

Preferably, the channels have a circular cross-section, to avoid edges and corners, or a rectangular or square cross-section, to simplify production, over their course. Nonetheless, other shapes are envisioned as well, such as trapezoid, triangular, parallelogram-shaped, etc.

The channel system comprises internal walls, and said walls are preferably provided with a hydrophobic and/or a hydrophilic coating. In some variations, a top section of the walls is provided with a hydrophobic coating, while one or more other sections are provided with hydrophilic coatings, preferably at least at a bottom section of the walls. The reverse is also possible.

Preferably, the internal volume of the channel system between the chip inlet and the first purification device is limited to at most 0.5 microliter, preferably at most 0.4 microliter, more preferably at most 0.3 microliter, even more preferably at most 0.25 microliter, or even at most 0.2 microliter, 0.15 microliter, 0.1 microliter, 0.05 microliter, or even lower, such as 0.025 microliter, 0.01 microliter, etc.

The reduction of transit volumes such as these, lowers the necessary amount of sample to be introduced in the microfluidic chip, as well as reducing potential losses of the proteins/protein complexes at internal surfaces of the channel system.

In further embodiments, the channel system further comprises at least one or more modules for protein purification or sample processing, which are selected from: additional purification devices, detection zones or systems for protein tracking, concentration devices for reducing the sample volumes and/or increasing protein amounts per volume, and/or on-chip valve systems.

In one embodiment, the microchip channel system thus comprises a purification device and comprises a (first) detection section suitable for detection of a label or marker in the sample, said detection section being positioned downstream from the purification device towards the chip outlet, preferably wherein the detection section is suitable for fluorescence detection.

The method as disclosed herein allows the use of labels or markers, that can be added to the original bulk sample from which the sample for a microfluidic chip is taken, to enable an operator to determine to which extent the sample has proceeded into the microfluidic chip. This is in particular useful in chips wherein internal valves, stoppers, or flow control constructions are provided, which allow the operator to control the flow of the introduced sample. Examples of such a marker or label are fluorescent labels, agents or dyes. These can easily be added to the sample without impacting the overall characteristics to be analyzed, and allow for an excellent detectability, even under the very small volumes and low optical pathlengths of sample used in microfluidics.

The detection section or zone or device, as used interchangeably herein, comprises a window through which the detection measurement of the medium therein can be analyzed, and specifically allows detection of the presence of the label or marker, typically automatically by a label or marker detection means, such as a fluorometer for fluorescence detection. With the term “medium” here, any fluid, potentially comprising solid components or not, is referred to, that is present in the detection section (sample, washing buffer, eluent, solvent, and/or mixtures thereof). By exposing the sample that has made it to the detection section to a light source or illumination means (with certain characteristics geared towards the label or marker), the detection means can then determine the presence, and specifically make an estimate on the amount or concentration, of the labels or markers in the detection section, which represent the concentration of the protein of interest and a control for the efficiency of the purification.

This modification allows the operator to track the progress of the sample in the channel system, knowing when the labeled sample first enters the detection section and when it has essentially left the detection section. For this, preset thresholds can be set, determining the minimal signal strength at which the sample is considered to enter the detection section, and the maximal signal strength under which the sample is considered to have fully left the detection section.

Based on this knowledge about the progress the sample has made, other actions may be performed, such as the timely introduction of a washing buffer, an eluent, steering of valves, pumps or other elements internal and/or external to the microfluidic chip, steering of a deposition means at the chip outlet, etc.

The detection section is preferably minimized in volume, to avoid any losses of the purified product to dead volume, adhesion to walls, and others. Preferably, the detection section is a cylindrical cavity, with the longitudinal axis perpendicular to the flow through the detection section. Most preferably, the openings (upstream and downstream) of the channel system that connect to the detection section are positioned on the intersections of a diameter of the circular cross-section of the cylindrical cavity, such that they are centered. Preferably, this is also the case with relation to the height at which they connect to the detection section. The choice for a circular cross-section is to match with most standard detection means, thereby keeping the volume of the detection section at a minimum. Additionally, the lack of internal edges reduces the risk of adhesion of the proteins/protein complexes of interest at such edges, where adhesive forces are higher.

Nonetheless, variations on the shape of the detection section are still possible, such as having a rectangular cross-section (via the z-axis) or others, for the sake of easier production, or other factors.

The detection section comprises internal walls, and said walls are preferably provided with a hydrophobic and/or a hydrophilic coating. In some variations, a top section of the walls is provided with a hydrophobic coating, while one or more other sections are provided with hydrophilic coatings, preferably at least at a bottom section of the walls. The reverse is also possible. In some embodiments, both the top and the bottom section are hydrophobic or hydrophilic, with at least some of the other internal walls of the detection section being the other type (respectively hydrophilic or hydrophobic). These configurations reduce the presence of air bubbles adhering to the walls, that could negatively impact transmission of light during the detection procedure.

In most embodiments, the detection will take place through an optical window at the top and/or at the bottom. In this light, it is important to avoid bubble formation and retention at these surfaces. It is found that bubbles are more prone to ‘stick’ to hydrophobic surfaces, therefore preferably these detection windows are hydrophilic.

Alternatively, other ‘side’ sections of the internal walls can be made hydrophobic, in order to gather the air bubbles there.

Preferably, the total internal volume of the detection sections is at most 500 nl, more preferably at most 250 nl, even more preferably at most 150 nl, and even more preferably at most 100 nl, or even 90 nl, 80 nl, 70 nl, 60 nl, 50 nl or lower. An example of this is a cylindrical detection section with a diameter of about 1.0 mm and a height of 0.1 mm, resulting in a total volume of about 78.5 nl.

Preferably, the (first) detection section is positioned downstream from the (first) purification device with an interstitial volume in the channels system of at most 0.5 microliter, preferably at most 0.4 microliter, more preferably at most 0.3 microliter, even more preferably at most 0.25 microliter, or even at most 0.2 microliter, 0.15 microliter, 0.1 microliter, 0.05 microliter, or even lower, such as 0.025 microliter, 0.01 microliter, etc.

As mentioned, the preferred embodiment provides for a detection section that allows fluorescence detection. This can be achieved by a choice in material that has a high transparency for light with a wavelength range around that for the fluorescent agent or dye that is added to the sample. Of course, this can vary strongly depending on the agent, typical agents often are centered in a certain range, such as between 250 to 700 nm, preferably centered towards between 300 to 600 nm or even between 400 nm and 550 nm. Another important factor is the absence of structural and surface defects and reducing the thickness of the material through which the fluorescence detection is performed. Potential material choices are one or more of polydimethylsiloxane (PDMS), glass, quartz, thermoset polyester (TPE), thermoplastic polymer, polystyrene (PS), polycarbonate (PC), poly-methyl methacrylate (PMMA), poly-ethylene glycol diacrylate (PEGDA), perfluorinated compounds (PFEP/PFA/PFPE), and/or others. In some embodiments, the chips are built up from separate layers, in which separate layers comprise different materials.

In conventional protein purification, even when analytical scales are applied, the amount of starting material needed for structure determination remains the bottleneck because the liquid volumes operated with during protein purification are in milliliters to liters range whereas only nano- to picolitre volumes of protein are needed for preparing cryo-EM grid. The microchip as presented herein allows to start from small amounts in microliter of sample, but even so, the fluid upon purification through the chip will dilute the protein present in the sample, which may be suboptimal for further analysis. So in a preferred embodiment, the channel system internally comprises a protein concentrator or protein concentration device, as used interchangeably herein, for intermediate concentration of the sample, preferably of the protein of interest, wherein said protein concentration device is positioned downstream of the purification device. If more than one purification device is present in the chip, each purification device may be followed with a downstream located protein concentrator and/or preceded upstream with a protein concentrator. Optionally a detection zone is present up- or downstream from said protein concentrator, depending on the desired protein detection measurement, after purification but prior to or after concentration.

In a preferred embodiment, the protein concentrator allows increasing protein concentration by at least 10-100 fold, and/or alternatively reduce the sample volume, with the further advantage that when placing the protein concentrator within the purification scheme of the chip, the final concentration of the protein of interest for spotting on the grid can be tuned. In combination with the on-chip valve systems disclosed herein, this technical setup of the chip, attached to a pumping system, valves and a controller allows for a one-device-from sampling to grid. Moreover, from a technical point of view, the on-chip concentrator allows adjusting protein concentration during cryo-EM grid preparation, therefore providing flexibility for optimization of the distribution of particles on cryo-EM grids and increasing the fraction of successfully prepared grids. Thus, protein structure determination will be faster and cheaper.

Preferably, the protein concentrator as used herein will be selected from a device or zone that applies any of the following principles:
a longitudinal electric field generated by passing current through a channel which is coated with an anionic bilayer, and controlled for its concentration effect by the voltage and the on-chip valve status of said protein concentrator present in the channel when the fluid flows through the concentrator;
a microcolumn or micro-device for ion exchange chromatography, thereby reducing the volume of the sample and increasing the protein of interest concentration by proceeding with its elution peak fraction (as detected in a detection zone downstream from said concentrator);
a semipermeable membrane for distinctive flow-through of proteins and/or compounds of the sample allowing to proceed with a higher amount of the protein of interest in the channeled fluid.

In a preferred embodiment, the channel system internally comprises an on-chip valve system downstream from the (first) purification device, and preferably downstream from the (first) detection section, towards the chip outlet. The channel internally comprises a first waste outlet, wherein the (first) on-chip valve system is arranged for switching a flow originating from upstream the channel system in view of the first on-chip valve system, said switching being between flowing towards the chip outlet and flowing towards the first waste outlet; or alternatively for retaining or holding (i.e. halting) the fluid for a short period of time as to control the incubation, concentration, or purification steps, for preventing the fluid from moving during incubation or concentration.

The incorporation of an internal on-chip valve system allows the purification process on the chip to be driven in a much more automated way than was previously possible. By providing external (or even internal) pumps or flow devices, washing buffers, eluents, and/or the sample can be introduced at the right moment, under the right conditions. By controlling the valve system, the flow can be diverted according to the needs, for instance allowing a washing buffer to flush out unbound proteins in the purification device from the sample, towards an outlet (dedicated or not), and then adapt the flow track allowing an eluent to elute the proteins/protein complexes of interest from the purification device, further down the channel system, towards the chip outlet.

As mentioned, the controlling of the valves in the valve system is preferably based on a detection signal from the preceding detection section.

In preferred embodiments, the total volume of the valve(s) is kept minimal, to avoid dead volume and adhesion of the purified product at such positions to walls of the chip. Preferably, the volume is at most 10.0 nl, more preferably at most 5.0 nl, even more preferably at most 2.5 nl, even more preferably at most 1.0 nl, or lower, such as 0.75 nl, 0.5 nl, etc. A tested example of such a valve had dimensions of 0.1 mm in each direction, resulting in a total volume of about 1.0 nl.

Preferably, the on-chip valve system is positioned downstream from the (first) purification device with an interstitial internal volume in the channel system therebetween of at most 1.0 microliter, preferably at most 0.5 microliter, more preferably at most 0.4 microliter, more preferably at most 0.3 microliter, even more preferably at most 0.25 microliter, or even at most 0.2 microliter, 0.15 microliter, 0.1 microliter, 0.05 microliter, or even lower, such as 0.025 microliter, 0.01 microliter, etc.

In a further preferred embodiment, the on-chip valve system (or valve system as used interchangeably herein) comprises at least one doormat valve, more preferably a slightly-open doormat (SOD) valve, and most preferably uses only such valves. The advantages are amongst others very high activation speed, operability via a vacuum actuation, and the possibility to be used in small-scale devices, such as a microfluidic chip.

In situations where there is only a single purification device, the valve system may also be responsible for dispensing the purified product onto a microscopy grid. It is positioned downstream from the first purification device, and comprises one or more separate valves, preferably at least two and more preferably at least three. These valves can be operably connected to a pump or other driving means for the valves. By opening and closing the necessary valves, the chip can be made to divert the flow of the medium in the channel system to the waste outlet. Once the eluted sample, with the purified protein/protein complex of interest, reaches the valve system, the valves can then be adapted, closing and/or opening certain valves, such that the waste outlet is no longer reachable, and the sample proceeds to the chip outlet from which it is dispensed onto a grid.

In a particular embodiment, the valve system that is positioned at the outlet of the chip comprises three separately controllable valves, each connected to a pump. In a particular embodiment, the first valve system comprises three separate valves, each connected to a pump. In another embodiment, the second valve system comprises three separate valves, each connected to a pump. Said valve system comprising three separately controllable valves is composed of a first valve, positioned in the most upstream position, after which the channel system splits into (at least) two different pathways, the first pathway leading to the waste outlet, the second to the chip outlet. The second and third valve are each positioned in one of the pathways, and are separately controllable, as is the first valve. By opening the first valve, the medium in the channel system can flow further down the channel system. In normal operation, where the sample with the purified product has not yet reached this point, the third valve is closed, blocking passage to the chip outlet, and the second valve is closed, allowing the medium to flow to and out of the waste outlet. The third valve is typically coupled to a pump or similar device that can provide a negative pressure, to suck the medium present in the channel system to the waste outlet. Once the sample with the purified product reaches the first valve (or slightly before or slightly after), the third valve is opened and the second is closed, such that the eluted purified product flows towards the chip outlet. Once the required amount is deposited onto the microscopy grid, it is then also possible to redraw (part of) the liquid dispensed product into the microfluidic chip, by closing the first valve and opening both the second and third valves. By applying a negative pressure at the waste outlet, the dispensed product can then be sucked back into the chip via the chip outlet, to the waste outlet to be expelled there. This system thus allows to control the dosing of dispensing fluid with purified protein or protein complex on the grid.

In embodiments where a further purification device is present in the chip after the first purification device, the first valving system can be simplified, in combination with the workings of a further valving system after the further purification device for instance, and/or in combination with pumps or similar means further downstream. The first valve system can then comprise a single valve (although more can of course be used) in a side-passage between the first and a second purification device, which extends from the main channel system therebetween. This side-passage leads to the first waste outlet. By opening the valve of the first valve system, and at the same time closing one or more valves further down the line, or other means to stop flow towards the chip outlet (for instance, a blocking means in the further purification device, although such a means functionally corresponds to a valve), the flow from the first purification device is diverted into the side-passage, and to the waste outlet. Again, preferably a negative pressure is provided at said waste outlet to ensure proper drainage of the medium. Once the purified product reaches the point of the side-passage (or again, slightly before or after), the valve of the first valve system can be closed, and flow can be restored towards the further purification device, and towards the chip outlet.

In a preferred embodiment, the channel system internally comprises a second purification device, preferably wherein the second purification device comprises at least one inlet for introducing and removing purification agents.

Preferably, the second purification device is of a different purification type than the first purification device, however alternatively, the second purification device may be of the same type as the first purification device, but usually with different or more specific purposes (such as, a different substance to exclude, different size for size exclusion, etc.). The second purification device is positioned downstream from the first purification device, and preferably downstream from the first detection section, towards the chip outlet.

The incorporation of multiple purification device in sequence on a single chip, allows a much more thorough purification process to take place, eliminating multiple undesired proteins or components in the sample, and increasing the efficiency of the purification process. This way, more specific purification devices, targeting specific substances, can be used that are more effective for said substances, while ensuring that further down the line, the other undesired substances are also removed from the sample. Especially by combining a multi-purification chip with a detection system and/or valve systems, both discussed in the present document, the invention enables a much higher degree of automation, a higher through-put volume, though using low sample amounts for purification, and more accurate dosing.

Specific combinations of different purification devices can vary depending on the intended use of the chip. One of the advantages is that certain columns can be easily modified to serve multiple purposes, as they can be loaded with different types of interacting agents, such as beads for size exclusion devices, which can be replaced after use.

Preferably, said second purification device is positioned downstream from the first purification device, with an interstitial volume of at most 5.0 μl, preferably at most 2.5 μl, more preferably at most 1.0 μl, even more preferably at most 0.75 μl, and even more preferably at most 0.5 μl. Even lower maximal interstitial volumes are further preferred, such as 0.4 μl, 0.3 μl, 0.25 μl, 0.2 μl, etc.

Preferably, said second purification device precedes the chip outlet with an interstitial volume of at most 5.0 μl, preferably at most 2.5 μl, more preferably at most 1.0 μl, even more preferably at most 0.75 μl, and even more preferably at most 0.5 μl. Even lower maximal interstitial volumes are further preferred, such as 0.4 μl, 0.3 μl, 0.25 μl, 0.2 μl, 0.1 μl, etc.

In a further preferred embodiment, the channel system internally comprises a second detection section, preferably suitable for fluorescence detection in a fluid medium in the second detection section, and/or preferably the second detection section being positioned downstream from the second purification device towards the chip outlet.

Under the same reasoning as for the (first) detection section, this allows the purification process to be monitored, and to be automated. Depending on the signal that is read from the (second) detection section, appropriate further actions can be initiated, such as starting or stopping of pumps, opening or closing of valves, introduction of a washing buffer, introduction of an eluent, etc. Again, the use of a detection section suitable for fluorescence detection is specifically advantageous, as it allows a fast, easy and very clear detection of the presence of labels or markers in the medium in the detection section, even under very low amounts present thereof. As the volume of the detection sections is to be kept minimal, the total amount of markers present is likewise low, leading to a much weaker detection signal. The applicants find that fluorescence provides for much stronger signals than typical labels or markers.

It should be noted that the same remarks as for the first detection section apply here, in relation to preferred embodiments in terms of volume, shape, distance to other features of the microfluidic chip (interstitial volumes) and other more specific embodiments.

In an even further preferred embodiment, the channel system internally comprises a second on-chip valve system downstream from the second detection section towards the chip outlet, and the channel system internally comprises a second waste outlet, wherein the second on-chip valve system is arranged for switching a flow originating from upstream the channel system in view of the second on-chip valve system, said switching being between flowing towards the chip outlet and flowing towards the second waste outlet.

As discussed previously for the first valve system, the presence of a second valve system, after the second purification device, can allow for a more delicate handling of the sample once introduced in the microfluidic chip, making sure that any precursor fluids to the sample (i.e., medium in the chip that proceeds the purified protein/protein complex downstream, such as fluids that were already present in the chip, or were washed out of purification devices prior to elution of the caught proteins/protein complexes of interest) are not dispensed onto a microscopy grid, but are instead diverted to a waste outlet. The same specifics as mentioned for the first valve system can be applied to the second valve system, for instance reduced dimensions, the use of slightly-open doormat (SOD) valves, etc.

Specifically, the second valve system can serve to dispense the purified product in exact, controlled doses. This can for instance be achieved as discussed for the first valve system, using the valve system comprising said three separately controllable valves. In this case, the second valve system comprises three separate valves, each connected to a pump. The first valve is positioned in the most upstream position, after which the channel system splits into (at least) two different pathways, the first pathway leading to the waste outlet, the second to the chip outlet. The second and third valve are each positioned in one of the pathways, and are separately controllable, as is the first valve.

By opening the first valve, the medium in the channel system can flow further down the channel system. In normal operation, where the sample with the purified product has not yet reached this point, the third valve is closed, blocking passage to the chip outlet, and the second valve is closed, allowing the medium to flow to and out of the waste outlet. The third valve is typically coupled to a pump or similar device that can provide a negative pressure, to suck the medium present in the channel system to the waste outlet. Once the sample with the purified product reaches the first valve (or slightly before or slightly after), the third valve is opened and the second is closed, such that the eluted purified product flows towards the chip outlet. Once the required amount is deposited onto the microscopy grid, it is then also possible to redraw (part of) the liquid dispensed product into the microfluidic chip, by closing the first valve and opening both the second and third valves. By applying a negative pressure at the waste outlet, the dispensed product can then be sucked back into the chip via the chip outlet, to the waste outlet to be expelled there.

In a preferred embodiment, the valves of the second valve system can be used to provide a way to stop flow towards the chip outlet, thereby allowing the first valve system to be simplified strongly, into a single valve which is present in a side-passage that leads to a waste outlet, as discussed previously. This side-passage splits off from the main channel of the channel system somewhere between the two purification devices (typically shortly after the first detection section, although in some embodiments, it can be positioned before the first detection section).

In all of the above, it should be considered that a third, fourth, etc. purification device can be provided further downstream. These may or may not be provided with a third, fourth, etc. detection section and/or valve system. The implementation of such further purification devices, detection sections and valve systems can be inferred from the information disclosed in this document for the first and second of said components and is considered to form an implicit part of the present application.

In a further preferred embodiment, the microfluidic chip comprises a second, or further chip inlet for introducing fluid media, in communication with the channel system at a position between the first purification device, and the second purification device, preferably at a position between the first detection section and the second purification device, and more preferably further upstream than the first on-chip valve system and further upstream than the first waste outlet.

Purification devices often work on the principle of catch and release. A substance is introduced (purification material or purification agent) that catches certain components (proteins or protein complexes) in the sample, but not other substances. These other substances are then washed out of the chip via a washing buffer, leaving behind only (or at least, mainly) the desired proteins or protein complexes. These are then subsequently released by introducing an eluent into the purification device, which flushes them free and further down the channel system. Although this could in theory be achieved by using the original chip inlet and the chip outlet (or another waste outlet) for introducing the washing buffer/eluent, it is much more convenient to approach this in a target way, by providing a second chip inlet between the first and second purification devices, such that the introduced liquid can skip the first purification device (and any potential remaining substances therein), and flows almost directly into the second purification device. This furthermore avoids contamination of the eluted product, with any remnants from upstream, that would be brought along with an eluent being introduced at the chip inlet. By providing one or more valves further upstream of the channel system, it can then be easily ensured that the introduced liquid at the second chip inlet flows downstream.

Most preferably, the second chip inlet is positioned directly in front of the second purification device, with a minimal interstice between the second chip inlet and the point where the channel system transitions into the second purification device. Such an interstice preferably has a volume of at most 0.1 microliters, more preferably at most 0.05 microliters, even more preferably at most 0.025 microliters.

In embodiments where a waste outlet is provided between the first and second purification device, the second chip inlet is preferably positioned further downstream than the waste outlet.

In a preferred embodiment, waste outlet(s) and/or chip inlet(s) are suitable to be connected to external channels or tubing, in which additional valves can be provided. Alternatively, these waste outlet(s) and/or chip inlet(s) can already comprise on-chip valves themselves. Both embodiments allow an easy way to incorporate the microfluidic chip in an overall control system, governing operation of the microfluidic chip, i.e., when, what and where a liquid (sample, eluent, washing buffer, washed out sample remains, purified sample) is introduced and/or removed.

In a preferred embodiment, the microfluidic chip is used for purification of one or more proteins and/or protein complexes of interest from a sample and subsequent deposition of the purified protein/protein complex onto electron-microscopy grids, prior to cryogenic electron microscopy (cryo-EM). The chip outlet is provided with a deposition means suitable for cryo-EM grid sample deposition, said deposition means being one of the following: capillary deposition means, drop-on-demand (DOD) deposition means, and pin printing deposition means; preferably wherein the deposition means is a capillary deposition means or wherein the DOD deposition means is a piezo-actuation deposition means.

Cryo-EM is an increasingly popular technique for structure determination of proteins and macromolecular complexes, allowing to determine the atomic resolution structures of macromolecular complexes that cannot be crystallized, amongst others. While the actual analysis only requires a very small volume of (purified) sample, the preparation thereof is difficult, as is the deposition onto a cryo-EM grid requires absolute precision. Furthermore, given the difficult procedure to purify the samples into a form amenable to cryo-EM, it is also crucial to maximize the use of the purified sample, using every femtoliter.

As such, the manner of deposition is also optimized in the improved microfluidic chip according to the invention.

In a particularly preferred embodiment, the deposition means is a capillary deposition means. Use of a capillary deposition means is advantageous as it allows a very thorough control of the process, making sure that the capillary can be emptied entirely, and is ideal for use in applications where the volume to be deposited is very small, as is the case here. Additionally, capillary deposition is relatively easy, compact enough to accomplish on a chip, and cheap.

Generally, cryo-EM deposition takes place by the formation of a purified sample liquid droplet at the end of the deposition means, in this case the end of the capillary deposition means, wherein a portion of the droplet is transferred onto a cryo-EM grid by contact. The excess volume can then be retracted, either by the same capillary deposition means, or by another capillary or another type of retraction means, and be disposed of.

The DOD deposition means was first used in printing technology, but has been applied in microscopy grid deposition in the past. It allows a very high control of deposited product with superb accuracy. DOD deposition typically falls into two separate methodologies, both using pressure waves which cause the drops to emerge from the deposition means. A first methodology using heat to generate the pressure waves is usually not applied in microfluidics as it is difficult to scale down and is inconvenient to use as it can affect the samples. The second methodology uses electrical voltages applied on piezoelectric materials (ceramics, crystal) to generate the pressure waves. These are deformed under the electrical voltage, thus creating an overpressure in the deposition means so the drop flows out. A further advantage is that it can easily be used in reverse, to retract the dispensed drop, by reversing the applied voltage, causing it to deform in the other direction and generating a negative pressure, thus sucking the drop back into the deposition means.

Similarly, pin printing deposition stems from printing technology as well. Pin printing deposition allows for low-volume depositions of a sample onto the grid using a pin. The purified sample is provided to the deposition means, and a metal pin is dipped into the sample to collect a predefined volume. The pin is preferably cooled down to dew point to prevent evaporation of the tiny droplet at its tip. The pin is moved to a predefined distance (for instance 10 μm) from a carrier surface of the grid, such that the sample forms a capillary bridge between the pin and grid. Once this bridge is formed, the pin is moved along the surface of the carrier. Capillary forces ensure that the liquid bridge follows the pin. If this parallel movement is sufficiently fast, a thin film will be deposited due to the viscous shearing on the liquid. The film thickness is determined by the relative speed of the pin moving over the carrier surface, the stand-off distance between pin and carrier substrate, the viscosity, surface tension, and surface properties of the pin and the carrier. The pin diameter itself will influence the range of stand-off distances one can use and the area one can write.

In a preferred embodiment, the purification device (the first and/or second) is one of the following types: affinity purification, ion exchange purification, dialysis purification, size exclusion purification, electrophoresis; preferably wherein the purification device is an affinity purification column or a size exclusion purification column.

In some embodiments, a purification device may be of multiple types, for instance a size-exclusion ion-exchange (SEIE) type, wherein ion-exchange particles are micro-encapsulated in or by a size-exclusion resin.

In some embodiments, the purification device of the type ion-exchange purification comprises cationic-exchange particles and/or anionic-exchange particles.

In cases where an affinity-type purification device is used (either as the first or second purification device), a number of further options exist therein, such as HisTrap, Strep-tag, antibody, or others.

In a second aspect, the invention relates to a system for electron microscopy, preferably cryogenic electron microscopy (cryo-EM), on protein samples, comprising:
at least one microfluidic chip according to the first aspect of the invention;
at least one illumination means and detection means for, preferably fluorescence, measurements on the microfluidic chip;
a pumping system adapted for operable connection to the microfluidic chip, and configured for controlling flow in the operably connected microfluidic chip;
a microscopy grid, preferably an electron microscopy grid, for holding fluid samples;
a control system, preferably a processor, which receives information on the measurements from the detection means, configured for controlling the pumping system at least based on said information, and configured for controlling the illumination and detection means.

Preferably, the control system is further configured for controlling any valves or valve systems on the microfluidic chip.

The total system is in its preferred embodiment aimed at use in cryo-EM. However, its application field is broader than that, as the potential for automation and keeping the entire purification process on a single chip, up to deposition, is a marked improvement over the prior art. By connecting a pumping system to the microfluidic chip, preferably hooked up to a number of different media containers (eluent, washing buffer, sample), the flow in the microfluidic chip can be controlled and steered. By having a control system govern the pumping system (and any valve system present) based on information from the detection means, minimal human supervision is required, as the control system can operate the entire procedure independently, from introduction of the sample up to deposition of the purified sample onto a microscopy grid.

Preferably, the above system is aimed at use in cryogenic electron microscopy (cryo-EM), and further comprises:
a cryogenic container, for a cryogenic coolant, preferably liquid ethane;
a cryogenic electron microscopy grid for holding the fluid samples; and
a transport system for moving the cryo-EM grid between a position for receiving a fluid sample from the microfluidic chip and the cryogenic container.

The control system is further configured for controlling the transport system.

By adding the above further components, the system becomes entirely autonomous and ready to fully prepare samples for analysis by a cryo-electron microscope, and its transport system can be further adapted (or alternatively, the system can comprise a second transport system to accomplish this) to be able to move the cryogenically treated samples on the cryo-EM grid to a cryo-electronic microscope.

In a third aspect, the invention relates to a method for on-chip purification and/or enrichment of one or more proteins and/or protein complexes of interest from a sample, preferably prior to cryogenic electron microscopy (cryo-EM), said method comprising the following steps:
providing a protein sample at a chip inlet of a microfluidic chip, preferably according to the first aspect of the invention, into a channel system of the microfluidic chip;
performing at least one purification step by means of a first purification device downstream of said chip inlet;
preferably dispensing the purified fluid protein sample onto a microscopy grid, preferably a cryo-EM grid, from a chip outlet via a deposition means downstream from the first purification device.

In a preferred embodiment, said protein sample is subjected to a first and a second purification by means of a first and second purification device present on said chip, preferably wherein the first and second purification preferably is of a different type (but alternatively wherein they are the same type), and wherein purification types are chosen from affinity purification, such as magnetic bead affinity purification, ion exchange purification, dialysis purification, size exclusion purification, or electrophoresis, or another type as known by the person skilled in the art.

In a preferred embodiment, a detergent is added to the sample prior, during or after introduction into the microfluidic chip. The applicant has found that this reduces the chance of the proteins/protein complexes adhering to the surface, which can impact the quality of the detection signal. Preferably, a detergent is added with a concentration relative to the sample of at most 1%, preferably at most 0.75%, 0.5%, and more preferably at most 0.4% or 0.3%. Most preferably the maximal concentration is 0.25% or even 0.2%. Preferably, a minimal concentration relative to the sample is added of at least 0.01%, preferably at least 0.025%, more preferably at least 0.05%, and most preferably at least 0.075% or even 0.1%.

In a preferred embodiment, the method comprises a step of measuring a fluorescence signal from the purified fluid protein sample at a first detection section downstream from the first purification column and upstream from the chip outlet, preferably wherein said step of measuring the fluorescence signal precedes a second purification step. Prior to introduction in the sample, the proteins or protein complexes of interest are labeled or marked via a fluorescent agent, such as a label, marker or dye. Preferred fluorescent agents comprise, amongst others Chromeo dyes, such as ChromeoP503, TAMRA dyes, BODIPY dyes, fluorescein, DyLight dyes, cyanine dyes, R-phycoerythrin (PE), lissamine rhodamine B, Texas Red, allophycocyanin (APC), Cy3.5, Cy5.5, Cy7, but the options are not limited thereto.

In a preferred embodiment, the method comprises a step of measuring a fluorescence signal from the purified fluid protein sample at a second detection section downstream from the second purification column and upstream from the chip outlet.

In a preferred embodiment, a first on-chip valve system is positioned downstream from the first purification device, and preferably downstream from the first detection section, towards the chip outlet, said first on-chip valve system configured to open and close, wherein closing the first on-chip valve system routes flow from the first purification device further downstream towards the chip outlet, and wherein opening the first on-chip valve system routes flow from the first purification device towards a first waste outlet; Preferably, a second on-chip valve system is positioned downstream from the second purification device, and more preferably downstream from the second detection section, towards the chip outlet, said second on-chip valve system configured to open and close, wherein closing the second on-chip valve system routes flow from the second purification device further downstream towards the chip outlet, and wherein opening the second on-chip valve system routes flow from the second purification device towards a second waste outlet.

### ASPECTS OF THE DISCLOSURE

The present invention relates in one aspect to a microfluidic chip for purification of one or more proteins and/or protein complexes of interest from a sample prior to cryogenic electron microscopy (cryo-EM), the chip comprising:
a) at least one chip inlet for receiving a fluid medium;
b) at least one chip outlet for dispensing fluid purified protein sample onto a cryogenic electron microscopy (cryo-EM) grid;
c) a channel system extending between the at least one chip inlet and the at least one chip outlet, said channel system internally comprising:
   i. a first purification device for purifying one or more proteins or protein complexes of interest from a sample, preferably wherein the first purification device comprises at least one inlet for introducing or removing purification agents.

A further embodiment discloses said microfluidic chip, wherein the channel system comprises a first detection section suitable for detection of a label or marker in the sample, said first detection section being positioned downstream from the first purification device towards the chip outlet, preferably wherein the first detection section is suitable for fluorescence detection.

Another further embodiment relates to said microfluidic chip, wherein the channel system internally comprises a first on-chip valve system downstream from the first purification device, and preferably downstream from the first detection section, towards the chip outlet, and the channel system internally comprises a first waste outlet, wherein the first on-chip valve system is arranged for switching a flow originating from upstream the channel system in view of the first on-chip valve system, said switching being between flowing towards the chip outlet and flowing towards the first waste outlet.

Another further embodiment relates to said microfluidic chip, wherein the channel system internally comprises a second purification device, preferably wherein the second purification device comprises at least one inlet for introducing and removing purification agents;

the second purification device being of the same or different purification type than the first purification device, and the second purification device being positioned downstream from the first purification device, and preferably downstream from the first detection section, towards the chip outlet.

Another further embodiment relates to said microfluidic chip according to the preceding embodiments, wherein the channel system internally comprises a second detection section, being positioned downstream from the second purification device towards the chip outlet, preferably suitable for fluorescence detection in a fluid medium in the second detection section. Another further embodiment relates to said microfluidic chip, wherein the channel system internally comprises a second on-chip valve system downstream from the second detection section towards the chip outlet, and the channel system internally comprises a second waste outlet, wherein the second on-chip valve system is arranged for switching a flow originating from upstream the channel system in view of the second on-chip valve system, said switching being between flowing towards the chip outlet and flowing towards the second waste outlet.

Another further embodiment relates to said microfluidic chip according to any of the preceding embodiments for purification of one or more proteins and/or protein complexes of interest from a sample and subsequent deposition of the purified protein/protein complex onto electron-microscopy grids, prior to cryogenic electron microscopy (cryo-EM), wherein the chip outlet is provided with a deposition means suitable for cryo-EM grid sample deposition, said deposition means being one of the following: capillary deposition means, drop-on-demand (DOD) deposition means, and pin printing deposition means; preferably wherein the deposition means is a capillary deposition means or wherein the DOD deposition means is a piezo-actuation deposition means.

Another further embodiment relates to said microfluidic chip for purification of one or more proteins and/or protein complexes of interest from a sample and subsequent deposition of the purified protein/protein complex onto electron-microscopy grids, prior to cryogenic electron microscopy (cryo-EM) with a capillary deposition means, wherein the on-chip valve system positioned nearest to the chip outlet comprises three separately controllable valves, wherein the first valve is positioned in the most upstream position, after which the channel system splits into at least two different pathways, the first pathway leading to the waste outlet, the second to the chip outlet, and the second and third valve are each positioned in one of the pathways.

Another further embodiment relates to said microfluidic chip according to any of the preceding embodiments, wherein the purification device comprises a solid pillar-based structure at the entrance and exit transition cross-section with the channel system, wherein the pillar-based structure has a height through the cross-section that is relatively smaller than the height of the purification device.

In a second aspect, the disclosure relates to a system for electron microscopy, preferably cryogenic electron microscopy (cryo-EM), on protein samples, comprising:
a) at least one microfluidic chip according to any of the preceding embodiments;
b) at least one illumination means and detection means for, preferably fluorescence, measurements on the microfluidic chip;
c) a pumping system adapted for operable connection to the microfluidic chip, and configured for controlling flow in the operably connected microfluidic chip;
d) a microscopy grid, preferably a cryogenic electron microscopy (cryo-EM) grid, for holding fluid samples;
e) preferably a cryogenic container, for a cryogenic coolant;
f) preferably a transport system for moving the cryo-EM grid between a position for receiving a fluid sample from the microfluidic chip and the cryogenic container;
g) a control system, preferably a processor, which receives information on the measurements from the detection means, configured for controlling the pumping system at least based on said information, and further configured for controlling the illumination and detection means and preferably also for controlling the transport system.

In a final aspect, the disclosure relates to a method for on-chip purification and/or enrichment of one or more proteins and/or protein complexes of interest from a sample, prior to cryogenic electron microscopy (cryo-EM), said method comprising the following step:
a) providing a protein sample at a chip inlet of a microfluidic chip, preferably according to any of the preceding claims 1 to 9, into a channel system of the microfluidic chip;
b) performing at least one purification step by means of a first purification device downstream of said chip inlet;
c) dispensing the purified fluid protein sample onto a cryogenic electron microscopy (cryo-EM) grid from a chip outlet via a deposition means downstream from the first purification device.

Another embodiment relates to said method, wherein said protein sample is subjected to a first and a second purification by means of a first and second purification device present on said chip, wherein the first and second purification preferably is of the same or a different type, and wherein purification types are chosen from affinity purification, ion exchange purification, dialysis purification, size exclusion purification, or electrophoresis. Another embodiment relates to said method which further comprises a step of measuring a fluorescence signal from the purified fluid protein sample at a first and/or second detection section upstream from the chip outlet.

Another embodiment relates to said method wherein a first on-chip valve system is positioned downstream from the first purification device, and preferably downstream from the first detection section, towards the chip outlet, said first on-chip valve system configured to open and close, wherein closing the first on-chip valve system routes flow from the first purification device further downstream towards the chip outlet, and wherein opening the first on-chip valve system routes flow from the first purification device towards a first waste outlet; and preferably wherein a second on-chip valve system is positioned downstream from the second purification device, and preferably downstream from the second detection section, towards the chip outlet, said second on-chip valve system configured to open and close, wherein closing the second on-chip valve system routes flow from the second purification device further downstream towards the chip outlet, and wherein opening the second on-chip valve system routes flow from the second purification device towards a second waste outlet.

Another embodiment relates to said method wherein dispensing the purified fluid protein sample onto a cryogenic electron microscopy (cryo-EM) grid from the chip outlet via a deposition means, is controlled by the on-chip valve system positioned nearest to the chip outlet and comprises three separately controllable valves, wherein the first valve is positioned in the most upstream position, after which the channel system splits into at least two different pathways, the first pathway leading to the waste outlet, the second to the chip outlet, and the second and third valve are each positioned in one of the pathways, so that dispension and withdrawing of the purified protein sample using negative pressure is obtained in the same capillary of the capillary deposition means.

The present invention will be now described in more details, referring to examples that are not limitative.

### EXAMPLES AND DESCRIPTION OF FIGURES

### Example 1. On-Chip Fluorescence-Based Protein Detection

Conventional purified protein detection involves measuring the absorbance signal at 280 nm UV. The UV absorbance signal is strong enough and is detectable only at millimeter-scale optical pathlength and tens-to-hundreds of μg/ml protein concentration. Under microfluidics setting with channel depth of 100 μm or less, an alternate detection schemes becomes imperative. Fluorescence-based protein detection is an excellent choice, since sensitivity of detecting fluorescent signal is 10 to 1,000 times higher than that of absorbance change. While proteins are intrinsically fluorescent due to the presence of aromatic amino acids (phenylalanine, tyrosine, and tryptophan), the content of aromatic amino acids is low, their fluorescence is weak and requires UV light for excitation, making detection not optimal. Therefore, proteins were labeled covalently with a fluorescent dye. ChromeoP503 was used for this purpose, which is a Py derivative fluorescent dye that possess an amine reactive pyrylium which reacts with primary amines in a protein forming a fluorescent adduct. The advantage of chromeo dye is that it becomes fluorescent only after covalently binding to amino acids. In addition, its emission is spectrally well separated from the excitation, making the detection of sub microliter quantities of protein robust.

The suitability of the dye for labelling was first verified on several test purified proteins which after labelling were ran on SDS-PAGE and protein positions were detected using fluorescent imaging.

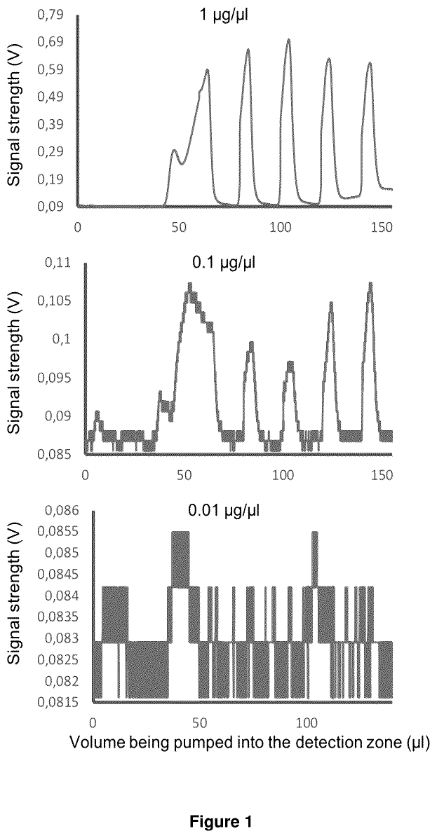

To determine the limit of detection of the experimental set-up, pure tagged BSA at different concentrations was pumped into the microfluidic chip alternately with buffer and was observed in the detection zone. FIG. 1 shows the minimum concentration of pure BSA that can be detected using our experimental set-up, with a plot of fluorescently tagged BSA at concentrations of 1 mg/ml, 0.1 mg/ml, and 0.01 mg/ml pumped alternately with buffer.

### Example 2. Integrated System for On-Chip Sample Purification and Application for Cryo-EM

The overall system according to the invention can be separated in several subsystems, and are discussed below for a specific configuration:

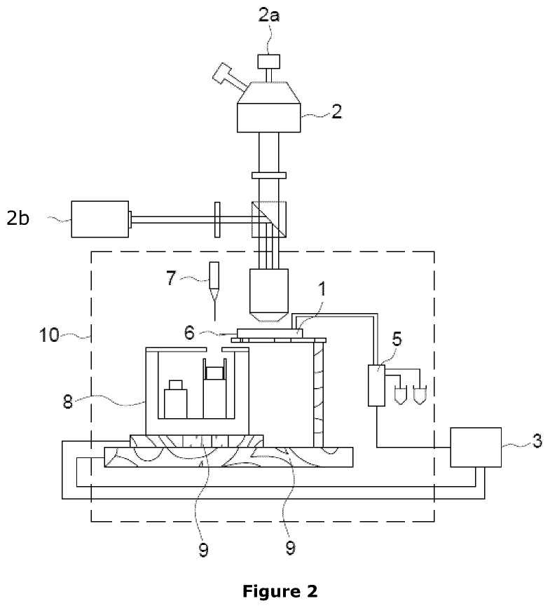

The whole set-up consists of 3 major modules: 1) Microfluidic chip with associated pumps and valves to control the flow, 2) an optics module consisting of a microscope, illumination and photodetection circuit for detection of the fluorescent signal, and 3) a cryo-plunger including Dewar with thermostatic ethane vessel and automated EM grid handling arm with the associated controls. The protein-handling parts of the setup are temperature controlled in the temperature range from 0 to 20° C. Automation of the processes is controlled using custom-built LabVIEW code. The schematic of the experimental set-up used is shown in FIG. 2.

In FIG. 2, a microfluidic chip (1) is shown positioned under a microscope (2), with photodetector (2a) and a light source (2b), for detecting the progress of the sample in the chip (1). The chip (1) is connected to a pumping system (5) for controlling the flow, and preferably valves, in the chip (1), and is capable of providing a number of fluids to the chip (1). An outlet provides the purified sample to a cryo-EM grid (6), which can be picked up by a plunging arm (7), and submerged in cryogenic coolant, such as liquid N₂, in a Dewar (8). A motion-control system (9) controls the movement of the plunging arm, cryo-chamber, and other components. The entire system is controlled by a processor or control system (3).

The entire system is comprised within a cooling chamber (10).

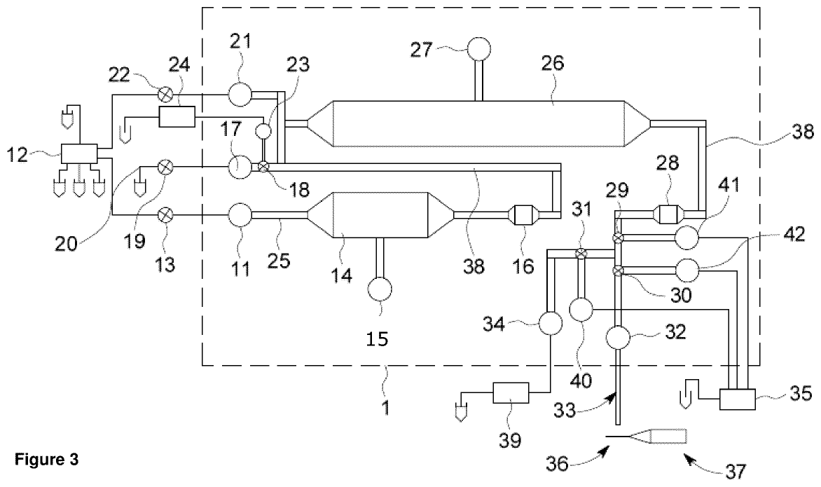

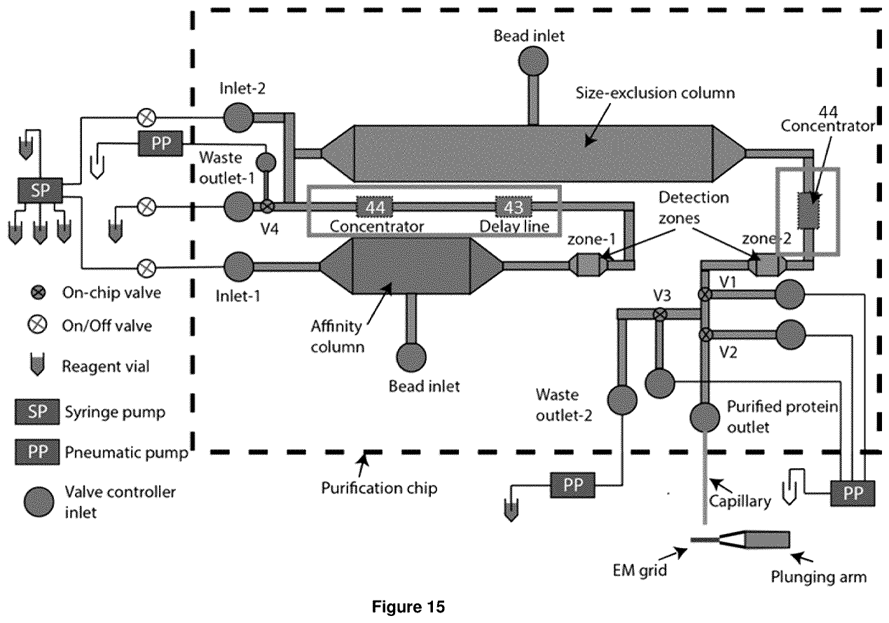

The microfluidic chip (1), shown in a possible embodiment in FIG. 3 and FIG. 15, comprises a chip inlet (11), to which a pumping system (12) can be connected. The (syringe) pumping system is provided with an off-chip valve (13) to control the flow of the sample into the chip (1). The chip inlet (11) continues via a channel system (25), which transitions to a first purification device (14), downstream of the chip inlet (11), in this case an affinity type purification column. It is provided with an inlet (15), which can be used for inserting a purification agent into the purification device (14), but may also be used as inlet and/or outlet for washing agents, eluent, etc. Further downstream from the first purification device (14), a detection zone (16) is provided through which the progress of the labeled sample in the microfluidic chip can be tracked. After the detection zone, a short delay zone (38) is present in the channel system of the chip (1), which continues to the second purification device (26), exemplified herein with a size-exclusion type column, again with an inlet/outlet (27). Prior to transitioning into the second purification device (26), the channel system splits off into a side-passage, which comprises an on-chip valve (18), and then splits up in two separate channels. The first is directed to a waste outlet (23) and connected to a pneumatic pump (24) for removing fluid from the channel system. The second separate channel is directed to an inlet (17) which in turn is connected to an off-chip valve (19) of the pumping system (12) and a reagent container (20).

Between the side-passage and the second purification device (26), another side-passage joins the main channel system, which serves as a second chip inlet (21) and is again connected to the pumping system (12), with an off-chip valve (22) therebetween.

After the second purification device (26), another delay zone (38) is present, leading up to the second detection zone (28). After said detection zone, the valving system at the chip outlet is present, also referred to herein as the valving system comprising ‘three separately controllable valves’ (29, 30, 31), specifically comprising a first valve (29), at which a side-passage is fluidly connected to the channel system, which ends in a valve controller inlet (41), which is operably connected to the pneumatic pump (35); from which the channel system then continues from the first valve (29) on towards a split in passages, of which the first passage of the split leads towards a further split towards a second valve (30) and a third valve (31), respectively. The second passage of the split is the continuation of the channel system and proceeds to a second valve (30), which is again joined by a side-passage ending in a valve controller inlet (42), which is operably connected to the pneumatic pump (35). The main channel system then leads onto the chip outlet (32), past the second valve (30) and the ensuing capillary deposition means (33). From said capillary deposition means (33), a purified sample deposition can be made onto an EM grid (36), which can be manipulated by a plunging arm (37).

At the third valve (31), the side-passage splits up in to subpassages, a first subpassage leading up to a waste outlet (34), which is connected to a pneumatic pump (39) and a collection volume. The second subpassage leads towards a valve controller inlet (40) which is again connected operably to the pneumatic pump (35).

The on-chip presence of said ‘three valve system’ or ‘three separately controllable valves’ (29, 30, 31), as used interchangeably herein, prevents that the fluid can move inside the chip even when the liquid is not being actively pumped through the chip. The latter is often observed due to relaxation of the pressure built up inside the chip and tubings. So by using a three valve system, the volume of the uncontrolled flow, which can go up to hundreds of nanoliters or larger, is significantly reduced or even avoided, and thus improves the accuracy of the control over the deposition of purified protein on the EM-grids.

So, the deposition of the fluid or purified sample on the (EM) grid or basically on any receiving material is obtained by the deposition means provided on the chip, preferably a deposition means for blotless grid preparation, as exemplified in FIG. 3 using a capillary deposition means, wherein said deposition means is preceded by a system of at least two, preferably three separately controllable valves, located upstream of said deposition means. So an advantage of applying on-chip valves upstream of said deposition means along with a liquid withdrawing mechanism by applying negative pressure during the purification method, enables the accurate flow control during the deposition of tens of nanoliters of protein solution on EM-grids. The presence of 3 valves allows for establishing direct connections between the capillary and the last chromatography column or capillary and waste outlet to which negative pressure is applied for liquid withdrawal. The presence of the valves on the chip increases the accuracy of flow control and the speed at which the flow direction can be changed.

In a further specific example as shown in FIG. 15, the microfluidic chip (1) further contains a delay line (43) present in a delay zone (38) for visualization of a complete elution profile or elution peak in the detection zone (16) prior to flow and injection of the protein sample into the second purification device (26), and allowing to perform multiple injections of sample fractions into the second purification device (26) by halting or retaining the flow. The latter is obtained by switching the on-chip valve (18) in the holding position, in line with the control on further on-chip valve(s) potentially present in the chip. Said delay line (43) thus allows to retain a larger volume of fluid in the channel delay zone (38) for a period of time required for complete elution from the first purification device (14) to be detected in the detection zone (16).

Additionally, as further outlined in Example 7, one or more protein concentration steps may be included in the on-chip purification method, and one or more protein concentration devices may therefore be integrated as for instance shown in the embodiment disclosed in FIG. 15. Specifically, the protein concentration device (44) may be implemented after the first purification device (14), and/or after the second purification device (26), preferably in the delay zone (38), the protein concentration device being positioned downstream of upstream of the detection zone, depending on the desired readout of the protein amount to be detected (prior or after protein concentration of the elution profile). In a preferred embodiment, the protein concentration device may be composed of an anionic bilayer coating present on the channel walls and is in conjunction with a closed on-chip valve.

The illumination and detection is accomplished by a Thorlab Cerna microscope fitted with 470 nm, 300 mW power LED (M470L3C5) in epi-fluorescence configuration was used to illuminate the detection zones. The resulting fluorescence emission was collected using the dichroic mirror and the emission filter at 570 nm onto a Silicon photodiode (SM1PD1A). The detected fluorescent signal was collected using a National Instruments Data Acquisition device USB-6001 and a custom-built biasing and amplification module. The collected data by the acquisition device were further passed onto the Labview VI, which will be subsequently processed to obtain the plot of detected signal as a function of volume of fluid that was being pumped into the purification chip.

The plunging system consists of mainly four parts namely plunging arm, cryo-chamber, humidity chamber, and motion-control system.

The plunging arm is composed of a Dumont self-closing precision tweezer (N7, 0.03 mm), a low-profile solenoid (2EC), stepper motor, and an electro-mechanical assembly that appropriately joins all the above components. The low-profile solenoid lets the tweezer do the picking and releasing of the grid through electronic actuation. The stepper motor allows the tweezer to have a rotational motion required for plunging in liquid ethane in a controlled way.

The cryo-chamber consists of a bath of liquid N₂ in which liquid ethane Dewar and a slot for placing the grid box are immersed. The cryo-chamber is 3D printed in polylactic acid (PLA) with isolation made form expanded polystyrene (EPS) which protects the liquid nitrogen from evaporating throughout the duration of experiment. The temperature of liquid ethane is measured and controlled using a thermocouple and a resistive heater. These are further controlled by Arduino board that communicates with LabVIEW.

The humidifier is a casing made of sponge and is supported through a structure. This surrounds the capillary and the EM grid. When the sponge is made wet, it creates a high humidity microenvironment that slows down the evaporation during deposition of protein solution during on the EM grid.

The motion-control system controls the movement of the plunging arm, cryo-chamber, and the humidifier both translationally and rotationally in the plane of the cryo-chamber with respect to the purification chip. These translational and rotational movements are controlled from LabVIEW.

The cooling chamber is the outermost covering of the experimental set-up encompassing the microfluidic chip, pumping system, and plunging system which provides the necessary low temperature environment for the proteins to be stable. The walls of the chamber are made with polyurethane foams and is being cooled using custom made hydraulic heat exchanger cooled by a Peltie chiller (AC 162, TE Technologies).

### Example 3. Purifying GFP Protein by Applying a Protein Sample on the Microfluidic Chip

The microfluidic chip as described in Example 2 was tested for the feasibility of purification using green fluorescent protein (GFP) as a test protein. GFP being intrinsically fluorescent, the fluorescent excitation was performed using the 473 nm LED and emission was observed using 505 nm filter and dichroic mirror. 30 μl of Cytoplasmic extract of GFP was pumped into the 5 μl affinity column of microfluidic chip and the unbound protein was washed away with wash buffer. The protein was subsequently eluted and when the peak was observed, 1 μl of this affinity purified protein was diverted into the 20 μl size-exclusion column. The eluent of the SEC column was collected in 2 μl vials and gel electrophoresis were performed. The gel was subsequently stained with silver stain to visualize the bands.

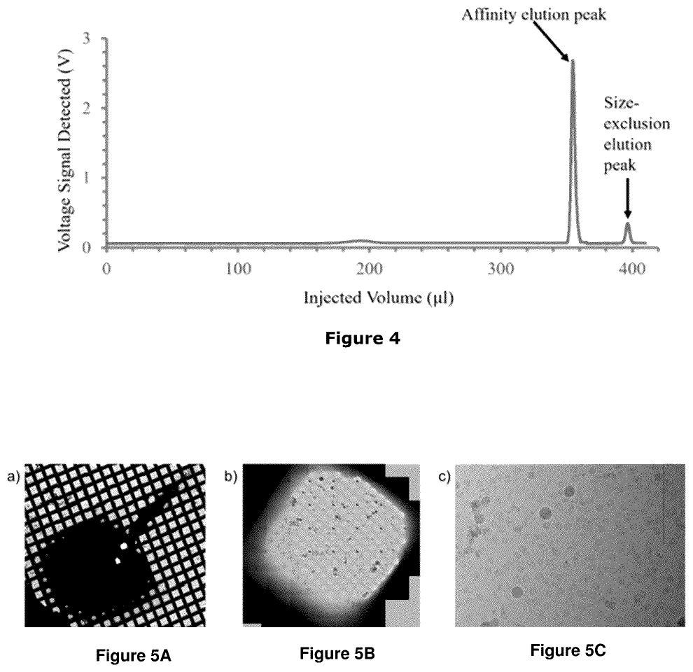

A similar experiment was performed but the eluent after the affinity purification was collected and gel electrophoresis was run. FIG. 4 shows the purification chromatogram observed during the GFP purification using the microfluidic chip and as detected by the first and second detection zones (28).

Further experiments were performed with different volumes of GFP cytoplasmic extract as the starting material to understand the limit of operation of the current experimental set-up.

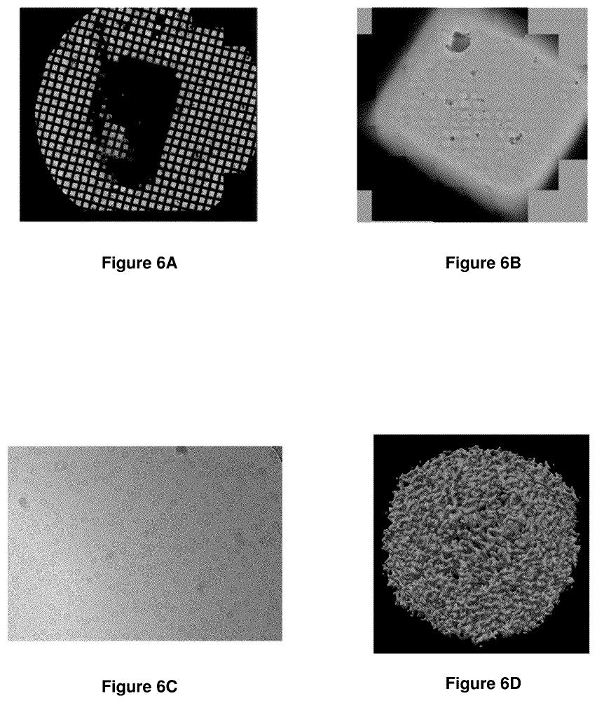

To test the plunging unit, purified GroEL and purified apoferritin were used as the test samples to be deposited onto the EM grids. The purified sample from the chip was brought onto the EM grid using a capillary attached to the chip. A small quantity of protein is deposited onto the grid and is sucked back into the capillary while the grid is in up and down motion. Immediately after that, the grid is plunged into the liquid ethane and transferred into the grid box stationed inside the liquid N₂ chamber. Single particle cryo-EM data were collected from the grid using high-resolution transmission electron microscope (TEM). For GroEL a total 1872 movies were collected and out of which 1156 good movies were used to extract 75,122 particles using crYolo. The particle images were further processed using Relion to obtain reconstruction at resolution of 4 Å. FIG. 5A shows the atlas of the grid, FIG. 5B shows one of outermost squares from where data collection was done and FIG. 5C shows a typical image of the particles from one of the holes. For Apoferritin, total about 1820 images were collected and out of which 976 good images were used for further processing using cryoSparc. About 108,000 particles were extracted and were used for reconstruction yielding a resolution of 2.6 Å. FIGS. 6A-D shows the representative images and the reconstructed apoferritin.

### Example 4. Complete Cryo-EM Structural Analysis from Cytoplasmic Extract to Cryo-EM Grid and Microscopy

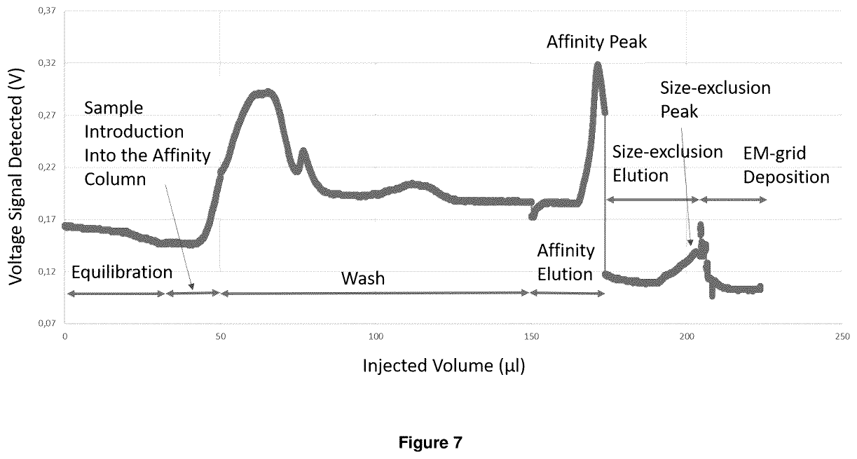

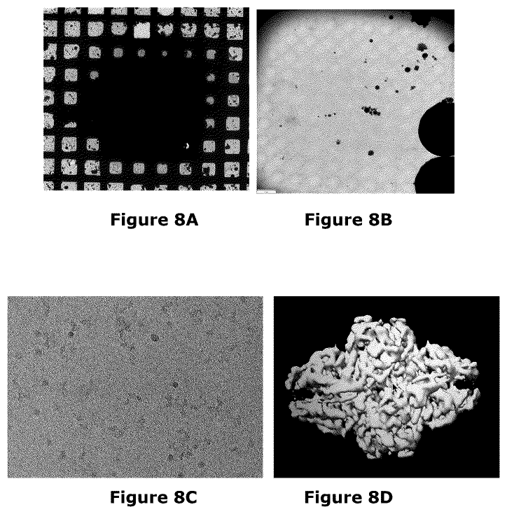

Cytoplasmic extract of Escherichia coli with expressed β-galactosidase was used to test the complete purification, EM grid preparation and plunging. 50 μl of crude sample of β-galactosidase was mixed with 2.5 μl of fluorescent dye and incubated for 1 hour as discussed above. 20 μl of this fluorescently tagged sample was pumped into the 5 μl affinity column that was filled Ni-NTA beads. The column was washed with wash-buffer and subsequently eluted using elution buffer. As the elution took place, the fluorescence signal was observed in the detection zone-1 (16). Once the peak in fluorescent signal was observed, this affinity purified protein of 1.5 μl volume was diverted into the size-exclusion column of 20 μl volume. Size-exclusion buffer was pumped to elute the sample from this column. The eluent once detected in the detection zone-2 (28), was diverted onto the EM grid using the capillary and three on-chip valves as shown in FIG. 3. About 100 nl of this purified protein was deposited and sucked back while simultaneously moving the capillary on the grid to ensure uniform spread of the protein. Once the deposition was complete the grid was plunged into the liquid ethane Dewar using the plunging arm that is holding the grid. In one session at least 8 EM grids can be plunged. The plunged grids were further analyzed using high-resolution TEM. A number of images was collected from one grid, out of which a subset was used for further processing. This yielded about 200,000 particles and finally yielding to a reconstruction of 3.2 Å resolution. FIGS. 7 and 8A-D shows the β-galactosidase purification chromatogram and few representative images of grid, squares, holes, and the final reconstruction respectively.

### Example 5. Purification of β-Galactosidase from a Single Colony Using a Particular Prototype of the Microfluidic Purification and Sample Prep System of the Present Invention

In Example 5, a microfluidic chip according to FIG. 9 is used, with an affinity column as the first purification device, and a SEC as the second. FIG. 9 shows the chip in multiple layers, as it is often constructed in three or more layers, which are affixed to each other by any number of processes or implements, such as glue, thermal bonding, lamination, and others. Alternative production methods, such as 3D printing are of course also available. Separate layers typically comprise different subcomponents of the chip (for instance, valves, purification device, channel system), with the outer layers closing the entire microfluidic chip off.

In this chip, the affinity column is of length 2 mm, width 2.5 mm and height 0.1 mm, amounting to a volume of 0.5 μl. SEC is having length 20 mm, width 2.5 mm and height 0.1 mm, amounting to a volume of 5 μl. Detection zones are circular with a diameter of 1 mm and height of 0.1 mm, amounting to a volume of about 80 nl. The critical part of the valve has a dimension of 0.1 mm×0.1 mm×0.1 mm, leading to a volume of 1 nl. It should be noted that these dimensions can be reduced further, to a volume of 1 nl for the affinity column, to 10 nl for the SEC and valves with a volume of only a few picolitres. After purification from affinity column, the purified protein has to pass through a volume of about 0.3 μl before entering into the SEC column. Similarly, after leaving the SEC column the purified protein has to pass through a volume of about 0.5 μl before it reaches the grid.

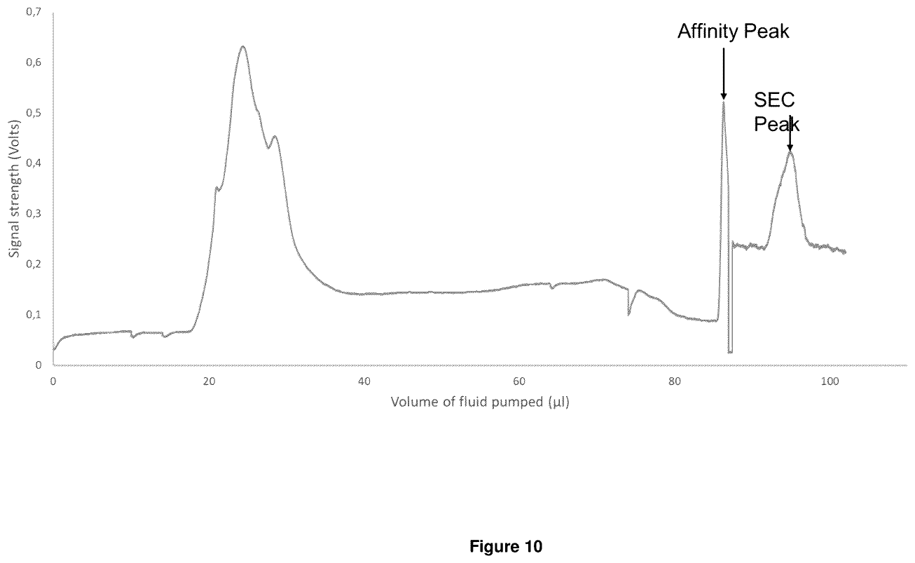

In the example, the amount of cytoplasmic extract loaded onto the chip for a given sample to dye ratio was evaluated by loading different amounts of cytoplasmic extract 20 μl, 4 μl, 1 μl and 0.5 μl onto the chip while maintaining the cytoplasmic extract to dye ratio of 40:1. The resulting signal strength is shown in FIG. 10 with respect to total volume introduced into the microfluidic chip for 4 μl of cytoplasmic extract loaded onto the chip. 1× and 10× amplifications were used during affinity and SEC respectively and 0.5 μl of affinity purified protein was diverted into SEC column.

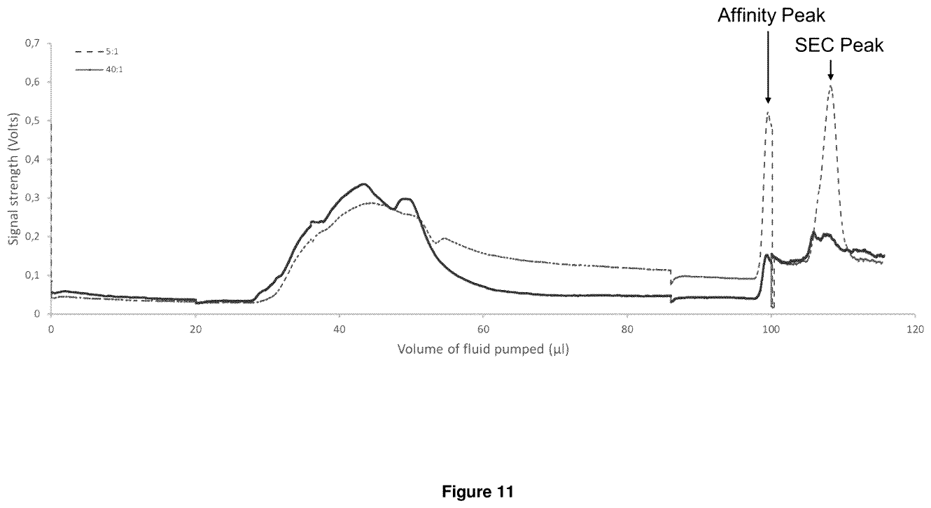

Cytoplasmic extract volume to fluorescent dye volume ratio is an important parameter to understand. Larger fluorescent dye volume for a given amount of cytoplasmic extract is supposed to increase the fluorescent signal after purification, presuming all the surface-exposed lysine residues in our protein of interest are not already tagged. This characterization was performed using the cytoplasmic extract from Escherichia coli expressing β-galactosidase and changed the ratio from 5:1 to 40:1. From the experimentally obtained plot, it is clear that as the dye volume was increased from 40:1 to 10:1 and then further to 5:1, the fluorescent signal after purification got increased. Again, 1× and 10× amplifications were used during affinity and SEC respectively and 0.5 μl of affinity purified protein was diverted into SEC column. This is shown in FIG. 11.

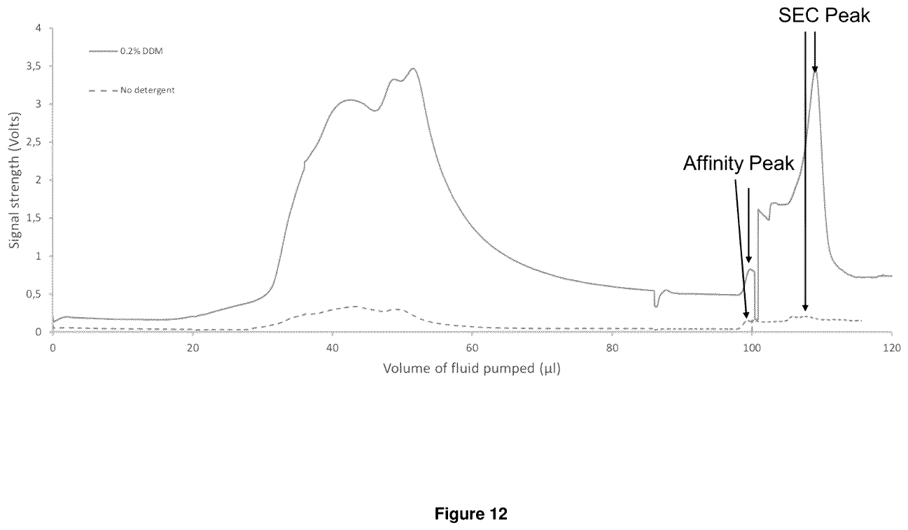

For the same situation, the effect of detergent [0.2% dodecyl-maltoside (DDM)] on protein adsorption inside the microfluidic chip was evaluated by adding 0.2% DDM to all buffers and pumping 4 μl of cytoplasmic extract onto the column and comparing it with the signal obtained in the earlier experiment without detergent. The chromatogram comparison shows the signal was greatly improved by the use of detergent. As before, 1× and 10× amplifications were used during affinity and SEC respectively and 0.5 μl of affinity purified protein was diverted into SEC column. This is shown in FIG. 12.

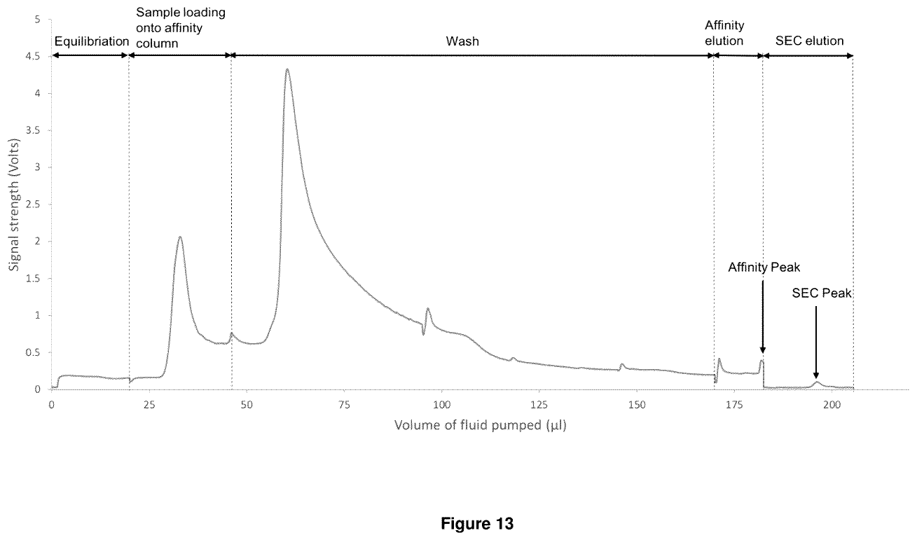

Subsequently, a test was run in relation to the purification of β-galactosidase from a single colony of cells to evaluate the possible lower limits of purification from cells. 40 μl of cytoplasmic extract was obtained from a single colony of cells. 35 μl of this cytoplasmic extract was mixed with 1 μl of fluorescent dye and 25 μl of this was loaded onto the chip. Again, 1× and 1× amplifications were used during affinity and SEC respectively and 0.5 μl of affinity purified protein was diverted into SEC column. The amount of β-galactosidase protein that is likely present in this amount of cytoplasmic extract that was pumped onto the chip, is about 0.5 μg (estimated based on reaction kinetics from spectrophotometer). The chromatogram is shown in FIG. 13. FIG. 16 shows the elution profile when eluted from the chip on SDS-PAGE.

The present example shows that a sample containing about 0.5 μg of β-galactosidase protein, was purified to high purity starting from a single colony extract to highly pure analyzable protein through processing on the microfluidic chip in about 1 hr. So not only heavily reduced need for protein amounts (as compared to standard laboratory purification in which about 150 mg of β-galactosidase is typically purified, at least 300,000 times lower protein amounts), but also the speed of the processing time from complex sample to purified analyzable product is significantly reduced (as a reference, conventional preparation of cryo-EM grids starting from protein purification using affinity and size-exclusion chromatography and grid plunging takes at least 3 to 5 hours, and longer for more challenging proteins, concluding that process time is 3-5 fold lower).

### Example 6. Complex-I Purification and Cryo-EM Grid Preparation

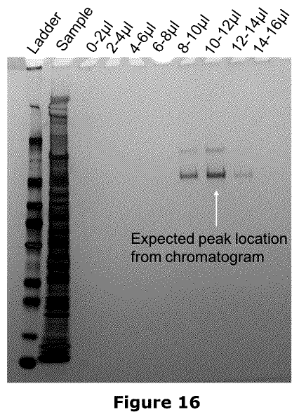

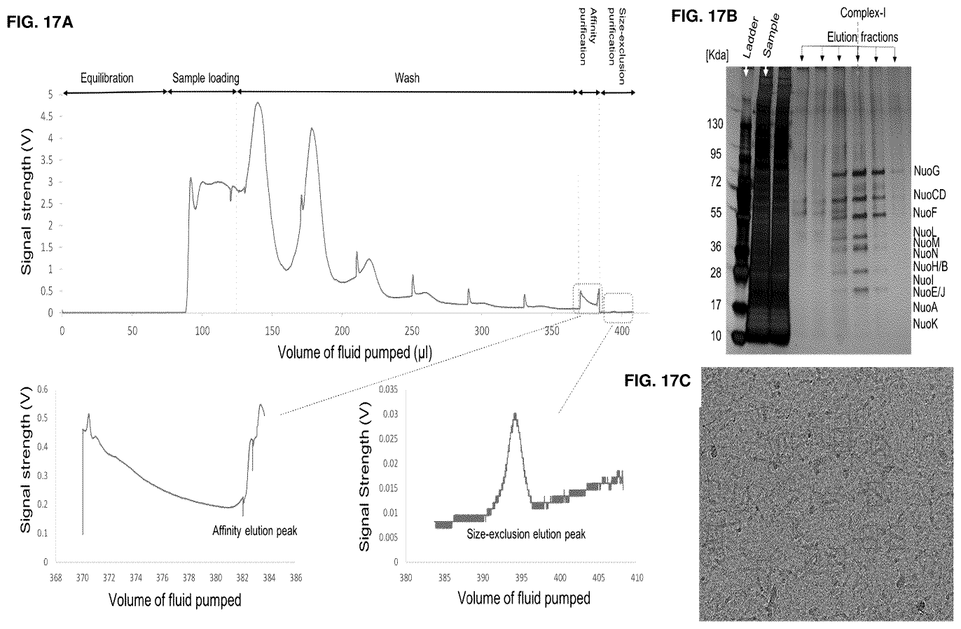

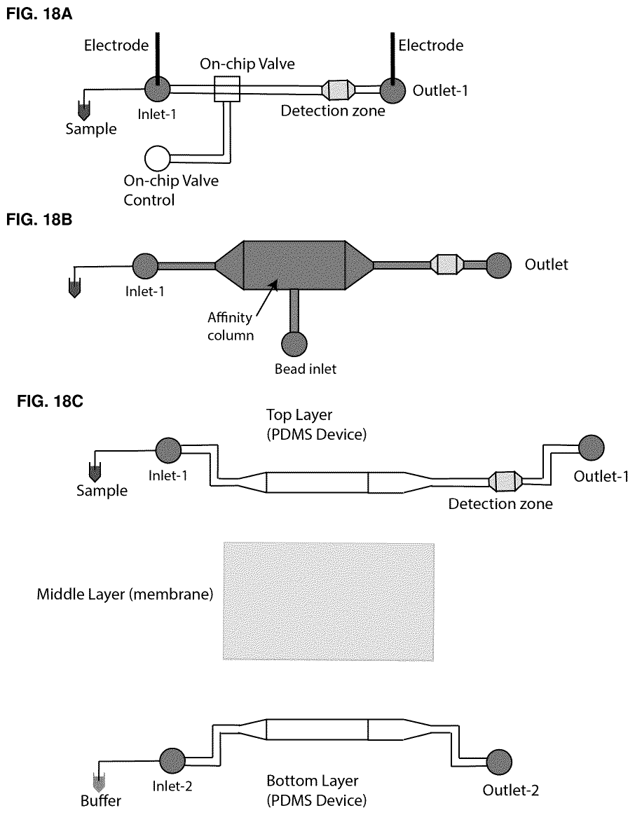

A volume of 50 μl of solubilized E. coli membranes containing about 0.4 μg of complex-I (Kolata and Efremov, 2021; eLife 10: e68710) was used for the protein purification using the methodology as presented herein and subsequently EM-grids were prepared. FIG. 17a shows the chromatogram obtained for the complete purification of E. coli complex-I using the developed microfluidic chip, FIG. 17b shows the SDS-PAGE analysis indicative that complex-I protein was isolated due to the presence of the stained bands corresponding to the different protein fragments constituting the complex. FIG. 17c shows an image of the complex-I particles in one of the acquired micrographs. This example demonstrates the efficiency of micro-purification using our device for a large fragile multi-subunit membrane protein complex expressed at low abundance.

### Example 7. Implementation of a Protein Concentration Device on the Microfluidic Chip for High-Resolution Structure Determination Using Cryo-EM

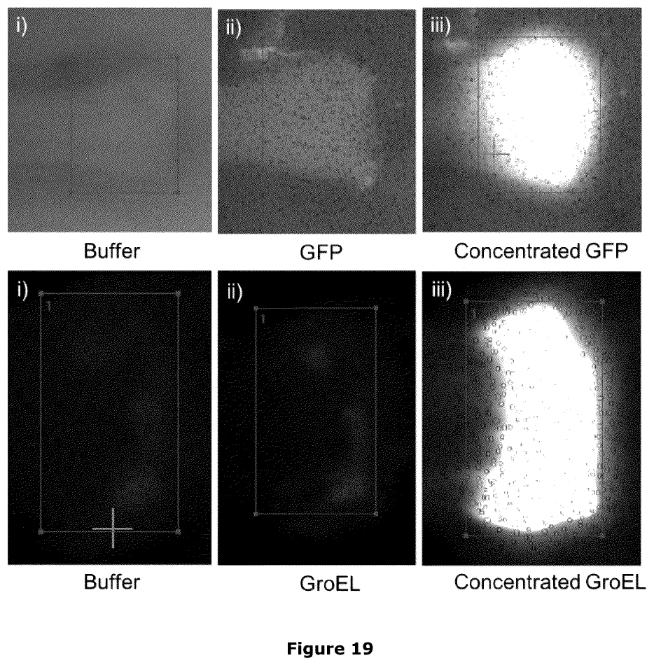

Protein concentration approaches based on the use of a longitudinal electric field and a normally closed valve have been experimentally verified for miniaturization and integration in microchips in the past (Kuo C H, Wang J H, Lee G B. A microfabricated CE chip for DNA pre-concentration and separation utilizing a normally closed valve. Electrophoresis. 2009; 30 (18): 3228-35), though only disclosed for use in DNA concentration, and not for protein concentration. We fabricated the microfluidic chip consisting of channel with an inlet and outlet and an on-chip valve. The channel region for protein concentration was coated with an anionic bilayer (by flowing and incubating 5% polybrene and 3% dextran sulfate solutions into the channel region) and rinsed with appropriate buffer, prior to loading the samples (100× diluted pure GFP for a 1st experiment and 1.5 mg/ml GroEL tagged with ChromeoP503 fluorophore for the 2nd experiment). The electric field was applied along the length of the channel and the concentration effect was observed near the valve due to a combination of electrophoretic effect and repulsion of negatively charged proteins in the nano channels of the closed valve. The valve downstream thereof is controlled pneumatically by applying pressure on the valve membrane making it closed or open depending upon applied pressure. During the concentration experiment, the valve was closed with 200 mbar pressure. The protein concentration effect was observed only when the valve was closed and wasn't observable with open valve. This process has led to concentrating by a factor of 14 for GFP and 25 for GroEL into a volume of 4 nl within the duration of 80 sec and 160 sec, respectively, under the application of mild electric field strengths of around 220 V/cm and 150 V/cm. The results are shown in FIG. 19.

The advantages of this method are that it enables us to control the extent of concentration based on the strength of electric field and duration applied. Even though this method of concentrating the protein adds additional complexity to the chip design due to the need to introduce electrodes, and possible bubble generation, the integration of this protein concentration step in the on-chip purification method is applied further.

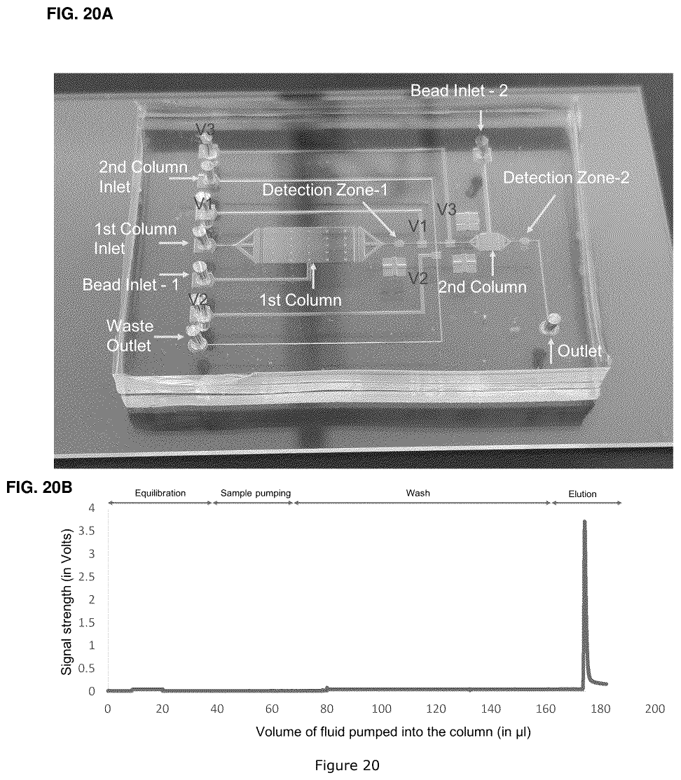

Secondly, the protein was analyzed for an on-chip protein concentration step using a micro-Ion Exchange Column. In this approach, a microfluidic chip consisting of two columns is made (using PDMS) as shown in FIG. 20a illustrating all relevant components for protein concentration as desired. The second column in this chip has a volume of 0.5 μl and was used for the concentration experiments. The second column was filled with Source 30Q strong anionic resin and pure GFP was used as protein sample to be concentrated. The signal that was detected in the detection zone when pure GFP was present and when the concentrated GFP are present are shown in FIG. 20b. This concentrator gave the average concentration of 3.5 times higher compared to that of pure GFP (in this example of loading 30 μl of pure GFP). The advantage of this method being its ability to provide variety of concentration factors depending upon the choice of column size, and amount of sample loading in addition to its simplicity. The possible drawback being the higher salt concentration in buffers, which can potentially hamper the usability of this method in the integration phase.

The present invention is in no way limited to the embodiments described in the examples and/or shown in the figures. On the contrary, methods according to the present invention may be realized in many different ways without departing from the scope of the invention.

1. A microfluidic chip for purification of one or more proteins and/or protein complexes of interest from a sample prior to cryogenic electron microscopy (cryo-EM), the chip comprising:
at least one chip inlet for receiving a fluid medium;
at least one chip outlet for dispensing fluid purified protein and or protein complexes;
a channel system extending between the at least one chip inlet and the at least one chip outlet, the channel system internally comprising a purification device for purifying one or more proteins or protein complexes of interest from a sample,
wherein the at least one chip outlet is provided with a deposition means suitable for cryo-EM grid sample deposition.

2. The microfluidic chip of claim 1, wherein the channel system further internally comprises one or more modules for protein purification, which are selected from:
a purification device for purifying one or more proteins or protein complexes of interest from a sample;
a detection section suitable for detection of a label or marker in the sample, said detection section being positioned downstream from a purification device towards the chip outlet;
a protein concentration device, for increasing the concentration of the protein in solution; and/or
an on-chip valve system downstream from a purification device and a waste outlet, wherein the on-chip valve system is arranged for switching a flow between flowing towards the chip outlet, flowing towards the waste outlet, or retaining the flow.

3. The microfluidic chip of claim 1, wherein the deposition means is controlled by at least two valves positioned in the channel system upstream of the deposition means.

4. The microfluidic chip of claim 1, wherein the deposition means suitable for cryo-EM grid sample deposition is selected from the group consisting of: capillary deposition means, drop-on-demand (DOD) deposition means, and pin printing deposition means.

5. The microfluidic chip of claim 1, wherein the channel system internally comprises
at least a first and a second purification device, and/or
at least one detection sections downstream from the purification device(s), and/or
at least one protein concentration device downstream from the purification device(s) and/or detection section, and/or
at least one on-chip valve systems downstream from the purification device(s) and/or from the detection section and/or from said protein concentration device,
wherein the second purification device is of the same or different purification type than the first purification device, and
wherein the second purification being is positioned downstream from the first purification device.

6. The microfluidic chip of claim 1, wherein further comprising at least one detection section is present and suitable for fluorescence detection.

7. The microfluidic chip of claim 1, wherein the protein concentration device is selected from the group consisting of:
an anionic bilayer coating present on the channel in conjunction with a closed on-chip valve either upstream or downstream of the coated channel, for electrophoresis-based protein concentration;
a semipermeable membrane for protein concentration; or
an ion exchange microcolumn.

8. The microfluidic chip of claim 1, for purification of one or more proteins and/or protein complexes of interest from a sample and subsequent deposition of the purified protein/protein complex onto electron-microscopy grids, prior to cryo-EM with a capillary deposition means, further comprising an on-chip valve system positioned proximal to the chip outlet, the on-chip valve system comprising first, second, and third comprises three controllable valves, wherein the first valve is positioned in the most upstream position, after which the channel system splits into at least first and a second pathways, the first pathway leading to a waste outlet, the second pathway to the chip outlet, the second valve being positioned in the first pathway and the third valve being positioned in in the third pathway.

9. The microfluidic chip of claim 1, wherein the purification device comprises a solid pillar-based structure at the entrance and exit transition cross-section with the channel system, wherein the pillar-based structure has a height through the cross-section that is smaller than the height of the purification device.

10. The microfluidic chip of claim 1, wherein the channel system further comprises at least one delay line positioned downstream from the purification device.

11. A system for electron microscopy, preferably cryo-EM, on protein samples, comprising:
at least one microfluidic chip of claim 1;
at least one illumination means and at least one detection means for measurements on the microfluidic chip;
a pumping system adapted for operable connection to the microfluidic chip, and configured for controlling flow in the operably connected microfluidic chip;
a microscopy grid for holding fluid samples; and
a control system which receives measurements from the detection means, configured for controlling the pumping system, and further configured for controlling the illumination and detection means.

12. A method for on-chip purification and/or enrichment of one or more proteins and/or protein complexes of interest from a sample, the method comprising:
providing a fluid protein sample at a chip inlet of a microfluidic chip and into a channel system of the microfluidic chip;
performing at least one purification step by means of a first purification device downstream of the chip inlet;
dispensing the purified or enriched fluid protein sample from a chip outlet via a deposition means downstream from the first purification device.

13. The method according to claim 12, wherein the dispensing is controlled by an on-chip valve system positioned proximal to the chip outlet and comprising first, second, and third separately controllable valves, wherein the first valve is positioned in the most upstream position, after which the channel system splits into at least first and second pathways, the first pathway leading to a waste outlet, the second to the chip outlet, the second valve being positioned in the first pathway and the third valve being positioned in the second pathway, so that dispension and withdrawing of the purified or enriched fluid protein sample using negative pressure is obtained in the same capillary of a capillary deposition means.

14. The method according to claim 12, wherein the fluid protein sample is subjected to a first and a second purification by means of a first purification device and second purification device present on the chip, wherein the first and second purifications are of the same or a different type, and wherein purification types are chosen from affinity purification, ion exchange purification, dialysis purification, size exclusion purification, and electrophoresis.

15. The method according to claim 12, wherein the method comprises a step of measuring a fluorescence signal from the purified or enriched fluid protein sample at a first and/or second detection section upstream from the chip outlet.

16. The method according to claim 14, wherein an on-chip valve system is positioned downstream from the first purification device, the on-chip valve system configured to open and close, wherein closing the on-chip valve system routes flow from the first purification device further downstream towards the chip outlet, and wherein opening the on-chip valve system routes flow from the first purification device towards a first waste outlet;
wherein closing the on-chip valve system routes flow from the second purification device further downstream towards the chip outlet, and wherein opening said on-chip valve system routes flow from the second purification device towards a second waste outlet.

17. The microfluidic chip of claim 1, wherein the purification device comprises at least one inlet for introducing or removing purification agents.

18. The microfluidic chip of claim 2, wherein the protein concentration device is positioned downstream from a purification device, and/or detection section.

19. The micro fluidic chip of claim 4, wherein the DOD deposition means is a piezo-actuation deposition means

20. The microfluidic chip of claim 5, wherein the second purification device is downstream from a detection section and/or a protein concentration device and/or on-chip valve system, towards the chip outlet
